(19) United States
(12) Patent Application Publication
(10) Pub. No.: US 2019/0374365 A1
(43) Pub. Date: Dec. 12, 2019

(54) ANKLE FOOT ORTHOSIS

(71) Applicant: INDUSTRIAL TECHNOLOGY RESEARCH INSTITUTE, Hsinchu (TW)

(72) Inventors: Tzong-Ming WU, Taipei City (TW); Ji-Bin Horng, Tainan City (TW); Tsung-Wen Tsai, New Taipei City (TW); Wai-Kwuen Choong, Selangor (MY)

(21) Appl. No.: 16/232,623

(22) Filed: Dec. 26, 2018

Related U.S. Application Data
(60) Provisional application No. 62/682,470, filed on Jun. 8, 2018.

(30) Foreign Application Priority Data
Dec. 19, 2018 (TW) 107145914

(51) Int. Cl. A61F 5/01

(52) U.S. Cl. CPC A61F 5/0127; A61F 2005/0165; A61F 2005/0137

(57) ABSTRACT

The disclosure relates to ankle foot orthosis including foot plate, lower leg piece, first and second connecting parts, and pin. The first connecting part is connected to the foot plate, the second connecting part is connected to the lower leg piece, the first connecting part has an engagement portion, the second connecting part has a groove and an opening connected to the groove, the first connecting part is pivotably engaged with the second connecting part, the pin is detachably disposed on the first connecting part of the first pivoting mechanism, and the second connecting part has a first stopper and a second stopper spaced apart from each other. The first pivoting mechanism has a first detachable state; when the first pivoting mechanism is in first detachable state, the engagement portion is located at the opening and is detachable from the groove via the opening.

### CROSS-REFERENCE TO RELATED APPLICATIONS

This non-provisional application claims priority under 35 U.S.C. § 119(a) on provisional patent application No(s). U.S. 62/682,470 filed in U.S.A. on Jun. 8, 2018, and on patent application No(s). 107145914 filed in Taiwan R.O.C. on Dec. 19, 2018, the entire contents of which are hereby incorporated by reference.

### TECHNICAL FIELD

The disclosure relates to an ankle foot orthosis, more particularly to an ankle foot orthosis with pivoting mechanism.

### BACKGROUND

For those who had suffered from cerebral palsy or stroke may have their muscles uncontrollably become too tense or relaxed and thus resulting in foot drop. If not properly treated, the muscles and ligaments in the injured ankle may gradually stiffen and lose function due to lack of exercise for a long time. Additionally, this type of patient would have other symptoms, such as eversion or clubfoot. Thus, suitable correction and rehabilitation are necessary based on the actual diagnosis results.

Ankle Foot Orthosis (AFO) is a medical aid for rehabilitation targeting on the aforementioned symptoms. In general, there are two types of ankle foot orthoses, one is pivotable, and the other is not pivotable.

Considering various requirements of rehabilitation and disabilities of the patient, the pivotable ankle foot orthosis is more convenient and flexible to use comparing to the unpivotable ankle foot orthosis. It is understood that the pivotal movement of the ankle foot orthosis is achieved by plenty of pieces assembled together, but current pivotal assembly is not allowed to be detached by the patient.

### SUMMARY

One of the embodiment provides an ankle foot orthosis including a foot plate, a lower leg piece, a first and second connecting parts, and a pin. The first connecting part is connected to the foot plate, the second connecting part is connected to the lower leg piece, the first connecting part has an engagement portion, the second connecting part has a groove and an opening connected to the groove, the engagement portion is located in the groove so that the first connecting part is pivotably engaged with the second connecting part, allowing the lower leg piece to be pivotably connected to the foot plate, the pin is detachably disposed on the first connecting part of the first pivoting mechanism, and the second connecting part has a first stopper and a second stopper spaced apart from each other so as to limit a movement range of the pin. The first pivoting mechanism has a first detachable state; when the first pivoting mechanism is in the first detachable state, the engagement portion is located at the opening and is detachable from the groove via the opening.

As the ankle foot orthosis discussed above, since one of the first connecting part and the second connecting part of the first pivoting mechanism has engagement portions, and the other one has groove and openings connected to the groove and allowing the engagement portions to pass through, the first connecting part and the second connecting part can be detached by directly removing the engagement portions from the groove via the opening. Therefore, the ankle foot orthosis can be detached in an efficient manner. In other words, the first connecting part and the second connecting part of the first pivoting mechanism can be assembled by directly engaging the engagement portions into the groove via the openings, thereby assembling the ankle foot orthosis in a fast manner. Therefore, the ankle foot orthosis can be assembled or disassembled by the wearer in a fast and convenient manner without using additional tools, which is convenient to wear and detach individual components for cleaning by the wearer for enhanced hygiene and comfort.

In addition, since the pin located between the first stopper and the second stopper is able to limit the degree of pivotal movement of the first connecting part and the second connecting part to each other so as to limit the pivotal movement of the lower leg piece relative to the foot plate, dorsiflexion and plantar flexion at the ankle are restricted.

### BRIEF DESCRIPTION OF THE DRAWINGS

The present disclosure will become better understood from the detailed description given hereinbelow and the accompanying drawings which are given by way of illustration only and thus are not intending to limit the present disclosure and wherein:

FIG. 1 is a perspective view of an ankle foot orthosis according to a first embodiment of the disclosure;

FIG. 2A to FIG. 2B respectively are a left side view and a right side view of the ankle foot orthosis in FIG. 1;

FIG. 3 is an exploded view of a first pivoting mechanism in FIG. 1;

FIG. 4 is an exploded view of a second pivoting mechanism in FIG. 1;

FIG. 5 is a side cross-sectional view of the first pivoting mechanism of the ankle foot orthosis according to the first embodiment of the disclosure when the first pivoting mechanism is in an assemblable state;

FIG. 6 is a schematic assembly diagram showing a pin in the first pivoting mechanism according to the first embodiment of the disclosure;

FIG. 7 is a side cross-sectional view of the second pivoting mechanism of the ankle foot orthosis according to the first embodiment of the disclosure when the second pivoting mechanism is in an assemblable state;

FIG. 8 is a schematic assembly diagram showing a second pin in the second pivoting mechanism according to the first embodiment of the disclosure;

FIG. 9 is a schematic diagram of the ankle foot orthosis according to the first embodiment of the disclosure as a lower leg piece is folded down;

FIG. 10 is a side cross-sectional view of the first pivoting mechanism of the ankle foot orthosis in FIG. 9;

FIG. 11 is a side cross-sectional view of the second pivoting mechanism of the ankle foot orthosis in FIG. 9;

FIG. 12 is a schematic diagram of the ankle foot orthosis according to the first embodiment of the disclosure when the lower leg piece is folded up;

FIG. 13 is a side cross-sectional view of the first pivoting mechanism of the ankle foot orthosis in FIG. 12;

FIG. 14 is a side cross-sectional view of the second pivoting mechanism of the ankle foot orthosis in FIG. 12;

FIG. 15 is a schematic diagram when an instep strap and a lower leg strap of the ankle foot orthosis according to the first embodiment of the disclosure are fastened in position;

FIG. 16 is a bottom view of the ankle foot orthosis in FIG. 15;

FIG. 17 is a schematic diagram showing the usage of the ankle foot orthosis in FIG. 15;

FIG. 18 is a side cross-sectional view of the first pivoting mechanism of the ankle foot orthosis in FIG. 15 during plantar flexion;

FIG. 19 is a side cross-sectional view of the first pivoting mechanism of the ankle foot orthosis in FIG. 15 during dorsiflexion;

FIG. 20 is a schematic diagram of the ankle foot orthosis according to the first embodiment of the disclosure as it is in a folded state;

FIG. 21A to FIG. 21B respectively are perspective views of an ankle foot orthosis according to a second embodiment of the disclosure at different viewing angles;

FIG. 22 is an exploded view of a first pivoting mechanism of the ankle foot orthosis according to the second embodiment of the disclosure;

FIG. 23 is a partially enlarged exploded view of the first pivoting mechanism in FIG. 21A;

FIG. 24 is a partial side cross-sectional view of the first pivoting mechanism in FIG. 21A;

FIG. 25 is an exploded view of a second pivoting mechanism of the ankle foot orthosis according to the second embodiment of the disclosure;

FIG. 26 is a side cross-sectional view of the first pivoting mechanism of the ankle foot orthosis according to the second embodiment of the disclosure when the first pivoting mechanism is in an assemblable state.

FIG. 27 is a schematic assembly diagram showing a pin in the first pivoting mechanism according to the second embodiment of the disclosure;

FIG. 28 is a side cross-sectional view of a second pivoting mechanism of the ankle foot orthosis according to the second embodiment of the disclosure when the second pivoting mechanism is in an assemblable state;

FIG. 29 to FIG. 30 are side cross-sectional views of the first pivoting mechanism and the second pivoting mechanism when a lower leg piece of the ankle foot orthosis according to the second embodiment of the disclosure is pivoted and placed on the foot plate;

FIG. 31 to FIG. 32 are side cross-sectional views of the first pivoting mechanism and the second pivoting mechanism when the lower leg piece of the ankle foot orthosis according to the second embodiment of the disclosure is pivoted and abut against lower leg of a patient;

FIG. 33 to FIG. 35 are schematic diagrams showing the movement of the pin of the first pivoting mechanism of the ankle foot orthosis according to the second embodiment of the disclosure as it is in one of insertion positions; and

FIG. 36 to FIG. 37 are schematic diagrams showing the movement of the pin of the first pivoting mechanism of the ankle foot orthosis according to the second embodiment of the disclosure as it is in another insertion position.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known main structures and devices are schematically shown in order to simplify the drawing.

In addition, the terms used in the present disclosure, such as technical and scientific terms, have its own meanings and can be comprehended by those skilled in the art, unless the terms are additionally defined in the present disclosure. That is, the terms used in the following paragraphs should be read on the meaning commonly used in the related fields and will not be overly explained unless the terms have a specific meaning in the present disclosure. Furthermore, in order to simplify the drawings, some conventional structures and components are drawn in a simplified manner to keep the drawings clean.

Further, the following embodiments are disclosed by the figures, and some practical details are described in the following paragraphs, but the present disclosure is not limited thereto. Furthermore, for the purpose of illustration, some of the structures and components in the figures are simplified, and wires, lines or buses are omitted in some of the figures. And the size, ratio, and angle of the components in the drawings of the present disclosure may be exaggerated for illustrative purposes, but the present disclosure is not limited thereto, and various modifications are allowed and can be made according to the following disclosure as long as it does not depart from the spirit of the present disclosure. Note that the actual size and designs of the product manufactured based on the present disclosure may also be modified according to any actual requirements.

On the other hand, in the following descriptions, the terms such as “first”, “second” and the like are merely used to distinguish components, and the components should not be limited by such terms. For example, within the scope of the disclosure, the “first” component in one embodiment may be called a “second” component, and the “second” component in one embodiment may be called a “first” component.

Further, the terms, such as “end”, “portion”, “part”, “area” and the like may be used in the following to describe specific components and structures or specific features thereon or therebetween, but are not intended to limit these components and structures. In the following, it may use terms, such as “substantially”, “approximately” or “about”; when these terms are used in combination with size, concentration, temperature or other physical or chemical properties or characteristics, they are used to express that, the deviation existing in the upper and/or lower limits of the range of these properties or characteristics or the acceptable tolerances caused by the manufacturing tolerances or analysis process, would still able to achieve the desired effect.

Furthermore, unless otherwise defined, all the terms used in the disclosure, including technical and scientific terms, have their ordinary meanings that can be understood by those skilled in the art. Moreover, the definitions of the above terms are to be interpreted as being consistent with the technical fields related to the disclosure. Unless specifically defined, these terms are not to be construed as too idealistic or formal meanings. The terms of the components in the disclosure are sometimes referred to in a more concise manner, depending on the requirements of the description, and should be understood by the reader.

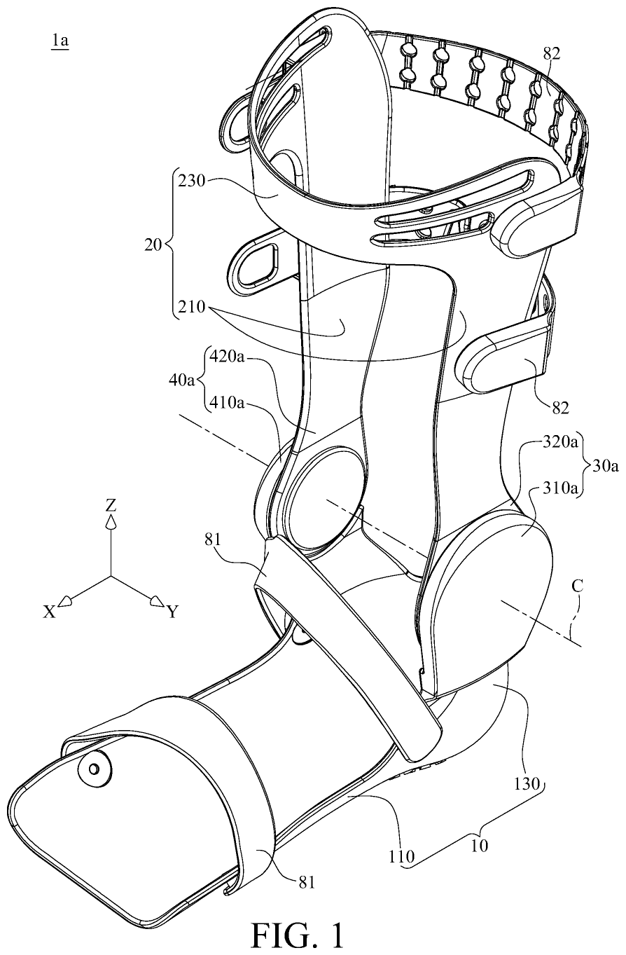

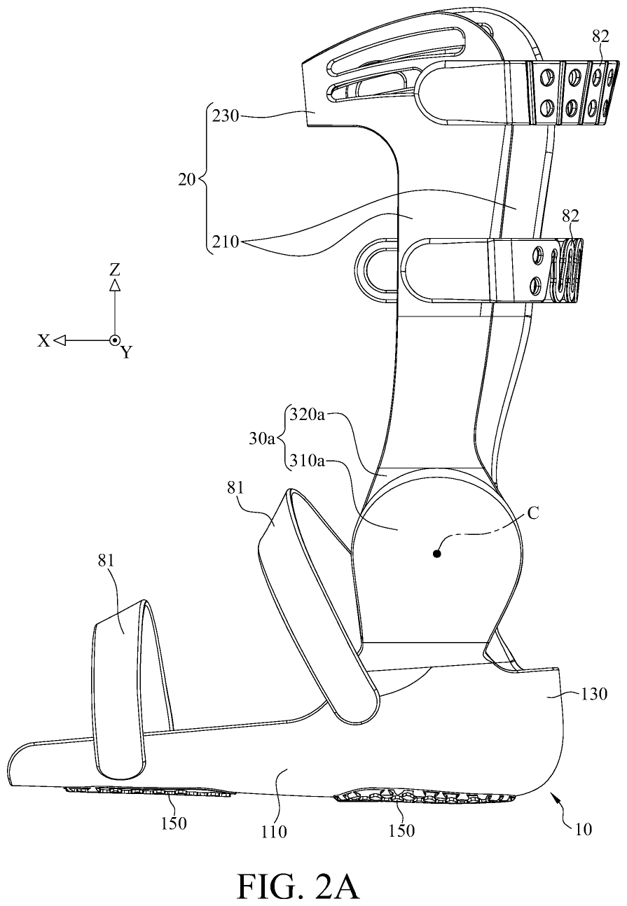

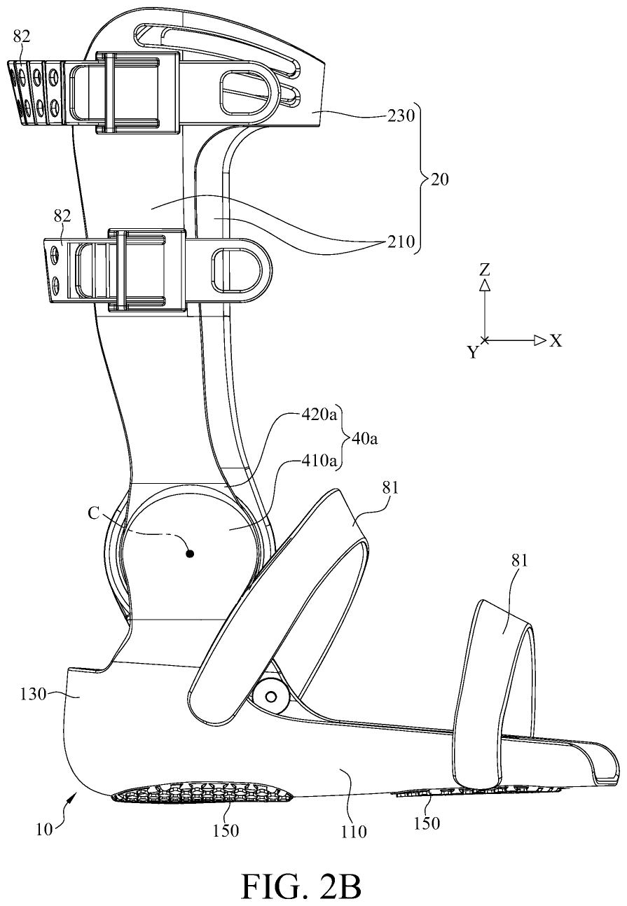

First, please refer to FIG. 1 and FIG. 2A to FIG. 2B; FIG. 1 is a perspective view of an ankle foot orthosis according to a first embodiment of the disclosure, and FIG. 2A to FIG. 2B respectively are a left side view and a right side view of the ankle foot orthosis in FIG. 1.

This embodiment provides an ankle foot orthosis 1a, more particularly an ankle foot orthosis whose joints are movable. It is explained that the ankle foot orthosis of this or other embodiments can be made by the cooperation of the three-dimensional (3D) scanning technology and 3D printing technology. In detail, the 3D scanner can scan and obtain the physical appearance information of lower limb and the foot, and then an ankle foot orthosis exclusively for the wearer will be created by the orthotic specialists based on the above physical appearance information. Therefore, the ankle foot orthosis will perfectly fit the lower limb and foot of the wearer. In addition, a further adjustment requested by the patient with respect to the physical appearance information is available added before being printed by the 3D printer. Further, the materials of the ankle foot orthosis include, for example but not limited to, polymer, such as nylon that can be repeatedly deformed and has the ability to return from deformation. Therefore, the above personalized processes make the ankle foot orthosis perfect-fitting, so that the ankle foot orthosis is not only cost-effective and high flexible but also helps considerably improve the rehabilitation. Specifically, for young-aged patient who are still in growing-up stage and need to keep buying new and bigger ankle foot orthosis for growing ankle, and for those who had suffered from stroke and need to buy different ankle foot orthoses with respect to different stages of rehabilitation, such personalized and digitalized processes also makes the ankle foot orthosis cost-effective for redesigning and manufacturing again.

In addition, since the 3D printing can build complex geometries such as lattice or honeycomb structures, the ankle foot orthosis of this or other embodiments may be strong in structural strength and lightweight, thereby reducing the weight while wearing, or the ankle foot orthosis may be made from a composite material so that the ankle foot orthosis is able to be made of a single piece but have hard and soft portions at the same time. For example, the ankle foot orthosis may have a soft inner layer and a hard outer layer or have a specific distribution of hard and soft portions that matches the injured ankle, thereby improving the practicality, comfort and durability. Also, with the help of the 3D printing technology, the ankle foot orthosis may have honeycomb structures or hollow features so as to make the ankle foot orthosis more lightweight and breathable.

Furthermore, since the ankle foot orthosis of this or other embodiments may be made by the above personalized processes, it is understood that, the following contents such as the relationship between the ankle foot orthosis and the wearer is only used for illustrating the suggestion for making the ankle foot orthosis depending on the wearer's injured ankle, but it is to be claimed that, the disclosure is not limited by the condition of the injured ankle, or the size and ratio shown in the drawings.

As shown in drawings, in this embodiment, the ankle foot orthosis 1a includes a foot plate 10, a lower leg piece 20, a first pivoting mechanism 30a, a second pivoting mechanism 40a, a plurality of instep straps 81, and a plurality of lower leg straps 82.

The foot plate 10 and the lower leg piece 20 are pivotablly connected to each other via the first pivoting mechanism 30a and the second pivoting mechanism 40a so that the foot plate 10 and the lower leg piece 20 can be pivoted relatively to each other along a central pivot axis C, thereby permitting the wearer's foot to perform plantar flexion and dorsiflexion.

Further, the foot plate 10 includes a support portion 110 and a heal cover 130. The support portion 110 is configured to support the wearer's foot. With respect to the design of the support portion 110, a supporting surface of the support portion 110 may be in a shape matching the bottom of the foot. Alternatively, there may be an additional insole disposed on the support portion 110 to fit the bottom of the foot, but the disclosure is not limited thereto.

The heal cover 130 is connected to the support portion 110 and may, but not limited to be integrally formed with the support portion 110. The heal cover 130 is configure to cover or hold the wearer's calcaneus or heal when the wearer's foot is on the support portion 110. In addition, with respect to the design of the heal cover 130, the heal cover 130 may be taller than or equal to the calcaneus, thereby covering the heal in a more comfortable manner and making the foot plate 10 fit the foot during walking so as to improve the comfort and the effect of the correction and rehabilitation. However, the heal cover 130 may be optional; in some other embodiments, the foot plate may not have the heal cover 130.

Two opposite ends of each instep strap 81 are respectively pivotably connected to two opposite sides of the support portion 110 (i.e., the left and right sides), and the instep straps 81 are used to cover part of the wearer's instep so as to hold the wearer's foot on the foot plate 10. In addition, in this or other embodiments, the two opposite ends of the instep strap 81 are detachably pivoted on the support portion 110, which is convenient to detach it for wearing. Additionally, the quantity of the instep straps 81 is not restricted and may be adjusted according to the actual requirements. In some other embodiments, there may be no instep strap 81; in such case, the shape of the support portion may be altered so that the support portion is able to be directly fixed onto the wearer's foot, but the disclosure is not limited thereto.

In addition, in this or other embodiments, the foot plate 10 may further include a plurality of anti-slip pads 150 disposed on a bottom surface of the support portion 110. The material or surface structure of the anti-slip pads 150 may help to increase the friction with the ground or shoes in order to improve the safety for the wearer to walk with the ankle foot orthosis 1a. The anti-slip pads 150 may be additionally attached to the bottom of the support portion 110 or integrally formed with the support portion 110 using 3D printing technology. In the latter case, it is understood that the anti-slip pads 150 are anti-slip structures directly formed on the bottom of the support portion 110.

The first pivoting mechanism 30a and the second pivoting mechanism 40a are, for example, located on the wearer's ankle. The first pivoting mechanism 30a includes a first connecting part 310a and a second connecting part 320a that are pivotably connected to each other, and the second pivoting mechanism 40a includes a third connecting part 410a and a fourth connecting part 420a that are pivotably connected to each other.

The first connecting part 310a of the first pivoting mechanism 30a and the third connecting part 410a of the second pivoting mechanism 40a are respectively connected to two opposite sides of the support portion 110 (i.e., the left and right sides) and are located on a side of the support portion 110 that is closer to the heal cover 130. In this or other embodiments, the first connecting part 310a of the first pivoting mechanism 30a, the third connecting part 410a of the second pivoting mechanism 40a, and the support portion 110 may, but not limited to be integrally formed. That is, the first connecting part 310a of the first pivoting mechanism 30a, the third connecting part 410a of the second pivoting mechanism 40a, and the foot plate 10 may, but not limited to be made of a single piece.

The second connecting part 320a of the first pivoting mechanism 30a is pivotably disposed on the inner side of the first connecting part 310a of the first pivoting mechanism 30a, the fourth connecting part 420a of the second pivoting mechanism 40a is pivotably disposed on the inner side of the third connecting part 410a of the second pivoting mechanism 40a, and the inner sides of the second connecting part 320a of the first pivoting mechanism 30a and the fourth connecting part 420a of the second pivoting mechanism 40a may be additionally provided with soft pads (not numbered) in order to improve the comfort while wearing. However, further introductions of the internal configurations of the first pivoting mechanism 30a and the second pivoting mechanism 40a are described later.

The lower leg piece 20 includes two upright parts 210 and a bridge part 230. The two upright parts 210 are respectively connected to the second connecting part 320a of the first pivoting mechanism 30a and the fourth connecting part 420a of the second pivoting mechanism 40a. In this or other embodiments, the second connecting part 320a of the first pivoting mechanism 30a, the fourth connecting part 420a of the second pivoting mechanism 40a and the two upright parts 210 may, but not limited to be integrally formed.

The bridge part 230 is connected to the two upright parts 210 and may, but not limited to be integrally formed with the two upright parts 210. With respect to the design of the lower leg piece 20, the two upright parts 210 are configured to be attached on sides of the patient's lower leg as the patient wears the ankle foot orthosis 1a, meanwhile, the bridge part 230 is designed to be in arc-shape to contact and hold the front side of the lower leg. By using the 3D printing technology, it is understood that shapes of the upright parts 210 and the bridge part 230 may perfectly fit that of the wearer's lower leg and thus improving the comfort and suitability.

The lower leg straps 82 are located on a side of the two upright parts 210 facing away from the bridge part 230, and two opposite ends of each lower leg strap 82 are respectively detachably connected to the two upright parts 210. In concrete, one end of the lower leg strap 82 may be detachably fixed to one of the upright parts 210, and the other end of the lower leg strap 82 may be adjustably and detachably engaged with engagement structure (not numbered) of the other upright part 210. As such, the lower leg straps 82 are detachable and adjustable with respect to the lower leg piece 20, and the wearer is allowed to adjust the tightness of the lower leg straps 82 by adjust the size of the space surrounded by the lower leg strap 82, two upright parts 210 and the bridge part 230. In addition, there may be an array of recesses (not numbered) formed on the lower leg straps 82 to make the lower leg straps 82 easier to be deformed. Further, the quantity of the lower leg straps 82 is not restricted and may be adjusted according to actual requirements. In some other embodiments, there may even be no lower leg strap 82 on the lower leg piece 20; in such a case, the shape of the upright parts may be altered so that the upright parts are able to be directly attached and fixed onto the wearer's lower leg, but the disclosure is not limited thereto.

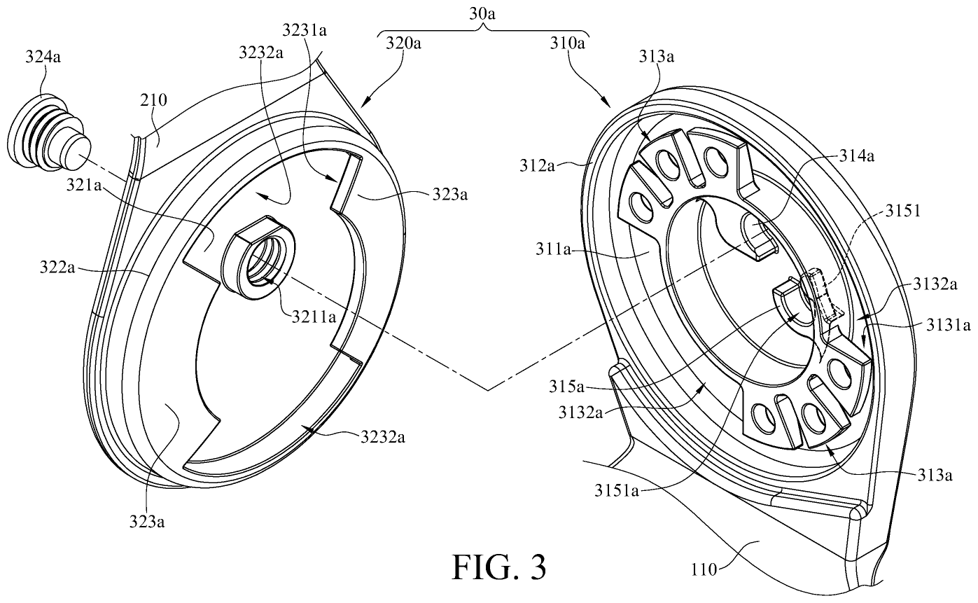

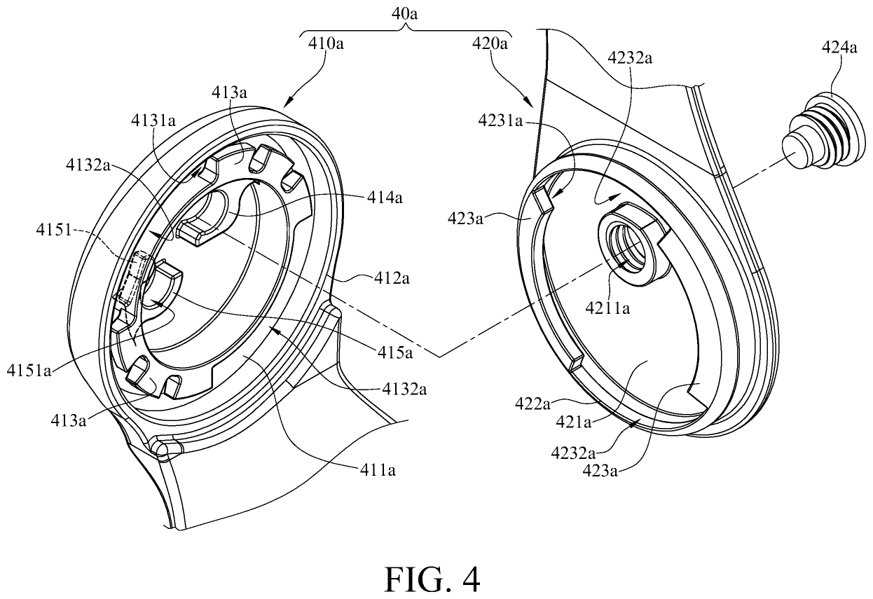

Then, the first pivoting mechanism 30a and the second pivoting mechanism 40a are introduced in more detail. Please refer to FIGS. 3 to 4; FIG. 3 is an exploded view of the first pivoting mechanism 30a, and FIG. 4 is an exploded view of the second pivoting mechanism 40a.

Firstly, as shown in FIG. 3, in this embodiment, the first connecting part 310a of the first pivoting mechanism 30a includes a first plate portion 311a, a first sidewall portion 312a, a plurality of first engagement portions 313a, a first stopper 314a, and a second stopper 315a.

The first plate portion 311a is connected to a side of the support portion 110, the first sidewall portion 312a protrudes toward the second connecting part 320a from the first plate portion 311a. The first engagement portions 313a are located in the area surrounded by the first sidewall portion 312a and are respectively located close to two opposite sides of the first sidewall portion 312a. The first engagement portions 313a are spaced apart from the first plate portion 311a so that a first groove 3131a is formed therebetween. The first engagement portions 313a are roughly arc-shaped and are spaced apart from each other in an arc-shaped direction so that two openings 3132a are formed between the first engagement portions 313a. The two openings 3132a are also roughly arc-shaped. In addition, in this or other embodiments, each of the first engagement portions 313a may have a through hole (not numbered), allowing the remaining materials in the first groove 3131a to be removed after the 3D printing process, but the disclosure is not limited thereto. The first stopper 314a and the second stopper 315a are located in the area surrounded by the first engagement portions 313a, and both of which are spaced apart from each other. In addition, as shown in FIG. 3, the second stopper 315a further has an elastic portion 3151. The elastic portion 3151 is connected to the second stopper 315a and extends toward the first stopper 314a from the second stopper 315a. The elastic portion 3151 is spaced apart from the first plate portion 311a and located above the first plate portion 311a. Thus, one end of the elastic portion 3151 away from the second stopper 315a is considered as a free end (not numbered).

The second connecting part 320a of the first pivoting mechanism 30a includes a second plate portion 321a, a second sidewall portion 322a, a plurality of second engagement portions 323a, and a pin 324a. The second plate portion 321a is connected to one of the upright parts 210. The second sidewall portion 322a protrudes toward the first connecting part 310a from the second plate portion 321a. The second engagement portions 323a are respectively located on two opposite sides of the second sidewall portion 322a and extend inward from a side of the second sidewall portion 322a facing away from the second plate portion 321a along radial directions. The second engagement portions 323a are spaced apart from the second plate portion 321a so that a second groove 3231a is formed therebetween. The second engagement portions 323a are roughly arc-shaped and are spaced apart from each other along arc-shaped direction so that two openings 3232a are formed between the second engagement portions 323a in arc-shaped direction. The two openings 3232a are roughly arc-shaped and directly connected to the second groove 3231a. The second plate portion 321a has an insertion hole 3211a located in the area surrounded by the second engagement portions 323a. The pin 324a can be detachably screwed or inserted into the insertion hole 3211a so as to protrude from the surface of the second plate portion 321a facing the first connecting part 310a. In addition, in other embodiments, the said pin may not have threads and may be able to be inserted into the insertion hole.

Then, as shown in FIG. 4, in this embodiment, the third connecting part 410a of the second pivoting mechanism 40a includes a third plate portion 411a, a third sidewall portion 412a, a plurality of third engagement portions 413a, a third stopper 414a, and a fourth stopper 415a.

The third plate portion 411a is connected to another side of the support portion 110, and the third sidewall portion 412a protrudes toward the fourth connecting part 420a from the third plate portion 411a. The third engagement portions 413a are located in the area surrounded by the third sidewall portion 412a and are respectively located close to two opposite sides of the third sidewall portion 412a. The third engagement portions 413a are spaced apart from the third plate portion 411a so that a third groove 4131a is formed therebetween. The third engagement portions 413a are roughly sector-shaped and are spaced apart from each other in arc-shaped direction so that two openings 4132a are formed between the third engagement portions 413a in arc-shaped direction. The two openings 4132a are roughly arc-shaped. In addition, in this or other embodiments, each of the third engagement portions 413a may have a through hole that allows the remaining materials in the third groove 4131a to be removed after the 3D printing process, but the disclosure is not limited thereto. The third stopper 414a and the fourth stopper 415a are located in the area surrounded by the third engagement portions 413a, and both of which are spaced apart from each other. In addition, as shown in FIG. 4, the fourth stopper 415a further has an elastic portion 4151. The elastic portion 4151 is connected to the fourth stopper 415a and extends toward the third stopper 414a from the fourth stopper 415a. The elastic portion 4151 is spaced apart from the third plate portion 411a and located above the third plate portion 411a. Thus, one end of the elastic portion 4151 away from the fourth stopper 415a is considered as a free end (not numbered).

The fourth connecting part 420a of the second pivoting mechanism 40a includes a fourth plate portion 421a, a fourth sidewall portion 422a, a plurality of fourth engagement portions 423a, and a pin 424a. The fourth plate portion 421a is connected to another upright part 210, and the fourth sidewall portion 422a protrudes toward the third connecting part 410a from the fourth plate portion 421a. The fourth engagement portions 423a are respectively located on two opposite sides of the fourth sidewall portion 422a and extend inward from a side of the fourth sidewall portion 422a facing away from the fourth plate portion 421a along radial directions. The fourth engagement portions 423a are spaced apart from the fourth plate portion 421a so that a fourth groove 4231a is formed therebetween. The fourth engagement portions 423a are roughly sector-shaped and are spaced apart from each other in arc-shaped direction. Thus, two openings 4232a are formed between the fourth engagement portions 423a in the arc-shaped direction. The two openings 4232a are roughly arc-shaped and directly connected to the fourth groove 4231a. The fourth plate portion 421a has an insertion hole 4211a located in the area surrounded by the fourth engagement portions 423a. The pin 424a can be detachably screwed or inserted into the insertion hole 4211a so as to protrude from the surface of the fourth plate portion 421a facing the third connecting part 410a.

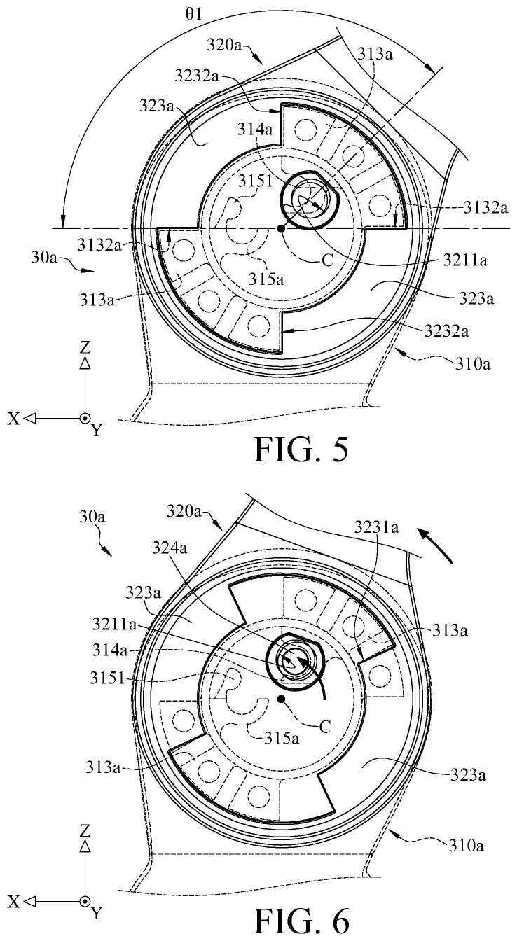

Then, the way to assemble the first pivoting mechanism 30a and the second pivoting mechanism 40a will be introduced below. Please refer to FIGS. 5 to 6; FIG. 5 is a side cross-sectional view of the first pivoting mechanism 30a when the first pivoting mechanism is in an assemblable state, and FIG. 6 is a schematic assembly diagram showing the pin in first pivoting mechanism. To assemble the first pivoting mechanism 30a, the first engagement portions 313a of the first connecting part 310a may be pivoted to the openings 3232a of the second connecting part 320a, and which is to simultaneously pivot the second engagement portions 323a of the second connecting part 320a to the openings 3132a of the first connecting part 310a. At this moment, the first pivoting mechanism 30a is in an assemblable state (i.e. detachable state) thereof. In this state, the first engagement portions 313a are allowed to be placed in the openings 3232a, meanwhile, the second engagement portions 323a are allowed to be placed into the openings 3132a, such that the first sidewall portion 312a of the first connecting part 310a will cover the second sidewall portion 322a of the second connecting part 320a. When the first pivoting mechanism 30a is in the assemblable state (i.e. detachable state) thereof, the insertion hole 3211a on the second connecting part 320a for the insertion of the pin 324a is not entirely located above the inner area between the first stopper 314a and the second stopper 315a. In this or other embodiments, the inner area between the first stopper 314a and the second stopper 315a is an area available for the movement of the pin 324a as the pin 324a is assembled in position, the other area is an outer area of the first stopper 314a and the second stopper 315a.

Then, the first connecting part 310a and the second connecting part 320a may be pivoted relatively to each other so as to engage the first engagement portions 313a into the second groove 3231a of the second connecting part 320a, meanwhile, the second engagement portions 323a are engaged into the first groove 3131a of the first connecting part 310a, and then the first connecting part 310a and the second connecting part 320a will be engaged with each other and the first pivoting mechanism 30a will be in an engaged state (or may be referred as an undetachable state). As shown in FIG. 6, the first connecting part 310a and the second connecting part 320a may be pivoted relatively to each other until the insertion hole 3211a is entirely located above the inner area between the first stopper 314a and the second stopper 315a. At this moment, the pin 324a can be inserted into the insertion hole 3211a and protrudes from the surface of the second plate portion 321a facing the first connecting part 310a to complete assembling the first pivoting mechanism 30a. At this moment, one end of the pin 324a is located between the first stopper 314a and the second stopper 315a, specifically, one end of the pin 324a is located between the elastic portion 3151 and the first stopper 314a. The relationships among the pin 324a with respect to the first stopper 314a, the second stopper 315a and the elastic portion 3151 during the operation will be introduced later.

It is understood that it is easy to disassemble the first pivoting mechanism 30a by reversing the above steps during the assembly. In detail, first is to remove the pin 324a or pull it out to a position that will not interfere with the first stopper 314a, the second stopper 315a and the elastic portion 3151, second is to pivot the first connecting part 310a and the second connecting part 320a to the state shown in FIG. 5 to allow the first engagement portions 313a to be removed from the openings 3232a (i.e., to remove the first engagement portions 313a from the second groove 3231a) and to allow the second engagement portions 323a to be removed from the openings 3132a (i.e., to remove the second engagement portions 323a from the first groove 3131a), and the last is to detach the lower leg piece 20 and the foot plate 10 from the first pivoting mechanism 30a.

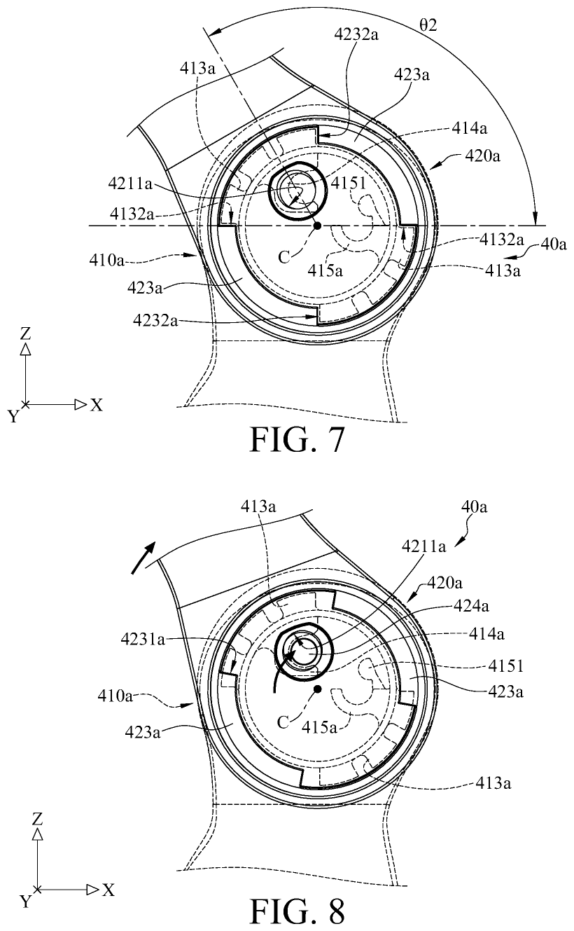

Then, please refer to FIG. 7 to FIG. 8; FIG. 7 is a side cross-sectional view of the second pivoting mechanism 40a when the second pivoting mechanism 40a is in an assemblable state, and FIG. 8 is a schematic assembly diagram showing the second pin in the second pivoting mechanism. Firstly, the third engagement portions 413a of the third connecting part 410a may be pivoted to the openings 4232a of the fourth connecting part 420a, and which is to simultaneously pivot the fourth engagement portions 423a of the fourth connecting part 420a to the openings 4132a of the third connecting part 410a. At this moment, the second pivoting mechanism 40a is in an assemblable state (i.e. detachable state) thereof. In this state, the third engagement portions 413a are allowed to be placed into the openings 4232a, meanwhile, the fourth engagement portions 423a are allowed to be placed into the openings 4132a, such that the third sidewall portion 412a of the third connecting part 410a will cover the fourth sidewall portion 422a of the fourth connecting part 420a. When the second pivoting mechanism 40a is in the assemblable state (i.e. detachable state) thereof, the insertion hole 4211a on the fourth connecting part 420a for the insertion of the pin 424a is not entirely located above the inner area between the third stopper 414a and the fourth stopper 415a. In this or other embodiments, the inner area between the third stopper 414a and the fourth stopper 415a is an available for the movement of the pin 424a as the pin 424a is assembled in position, the other area is an outer area of the third stopper 414a and the fourth stopper 415a.

Then, the third connecting part 410a and the fourth connecting part 420a may be pivoted relatively to each other so as to engage the third engagement portions 413a into the fourth groove 4231a of the fourth connecting part 420a, meanwhile, the fourth engagement portions 423a are engaged into the third groove 4131a of the third connecting part 410a, and then the third connecting part 410a and the fourth connecting part 420a will be engaged with each other and the second pivoting mechanism 40a will be in an engaged state (may also referred as an undetachable state). As shown in FIG. 8, the third connecting part 410a and the fourth connecting part 420a may be pivoted relatively to each other until the insertion hole 4211a is entirely located above the inner area between the third stopper 414a and the fourth stopper 415a. At this moment, the pin 424a can be inserted into the insertion hole 4211a and protrudes from the surface of the second plate portion 421a facing the third connecting part 410a, and one end of the pin 424a is located between the third stopper 414a and the fourth stopper 415a. And the relationship among the pin 424a with respect to the third stopper 414a, the fourth stopper 415a and the elastic portion 4151 during the operation will be introduced later.

It is understood that it is easy to disassemble the second pivoting mechanism 40a by reversing the above steps during the assembly. In detail, first is to remove the pin 424a or pull it out to a position that will not interfere with the third stopper 414a, the fourth stopper 415a and the elastic portion 4151, second is to pivot the third connecting part 410a and the fourth connecting part 420a to the state shown in FIG. 7 to allow the third engagement portions 413a to be removed from the openings 4232a (i.e., to remove the third engagement portions 413a from the fourth groove 4231a) and to allow the fourth engagement portions 423a to be removed from the openings 4132a (i.e., to remove the fourth engagement portions 423a from the third groove 4131a), and the last is to detach the lower leg piece 20 and the foot plate 10 from the second pivoting mechanism 40a.

As discussed above, the assemble and disassemble of the first pivoting mechanism 30a and the second pivoting mechanism 40a can be completed without additional tool. Therefore, the ankle foot orthosis 1a can be assembled or disassembled in a fast and convenient manner, which is convenient, time-saving, easy to be detached individual components for cleaning.

Additionally, it is explained that the assemble/disassemble angles of the first pivoting mechanism 30a and the second pivoting mechanism 40a are different. In detail, please compare FIG. 5 and FIG. 7, as shown in FIG. 5, when the first pivoting mechanism 30a is in the assemblable state (i.e., the detachable state), an imaginary line passing through the insertion hole 3211a and the central pivot axis C has a first angle θ1 at the ground, the first angle θ1 may be considered as the current angle between the foot plate 10 and the lower leg piece 20; as shown in FIG. 7, when the second pivoting mechanism 40a is in the assemblable state (i.e., the detachable state), an imaginary line passing through the insertion hole 4211a and the central pivot axis C has a second angle θ2 at the ground, the second angle θ2 may be considered as the current angle between the foot plate 10 and the lower leg piece 20, but the second angle θ2 is different from the said first angle θ1. That is to say, when the foot plate 10 and the lower leg piece 20 are pivoted to a specific angle that allows the first pivoting mechanism 30a to be assembled and disassembled, the second pivoting mechanism 40a is not allowed to be assembled and disassembled. This helps the user to assemble or disassemble the first pivoting mechanism 30a and the second pivoting mechanism 40a one at a time and helps to avoid the first pivoting mechanism 30a and the second pivoting mechanism 40a from falling apart at the same time during the disassembly thereof.

Then, the following will explain how to wear the ankle foot orthosis 1a and further explain the relationships among the internal components of the first pivoting mechanism 30a and the second pivoting mechanism 40a.

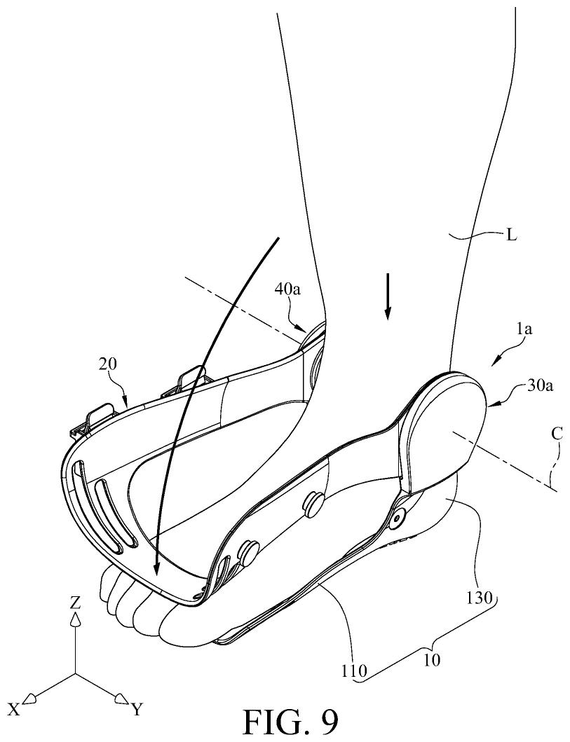

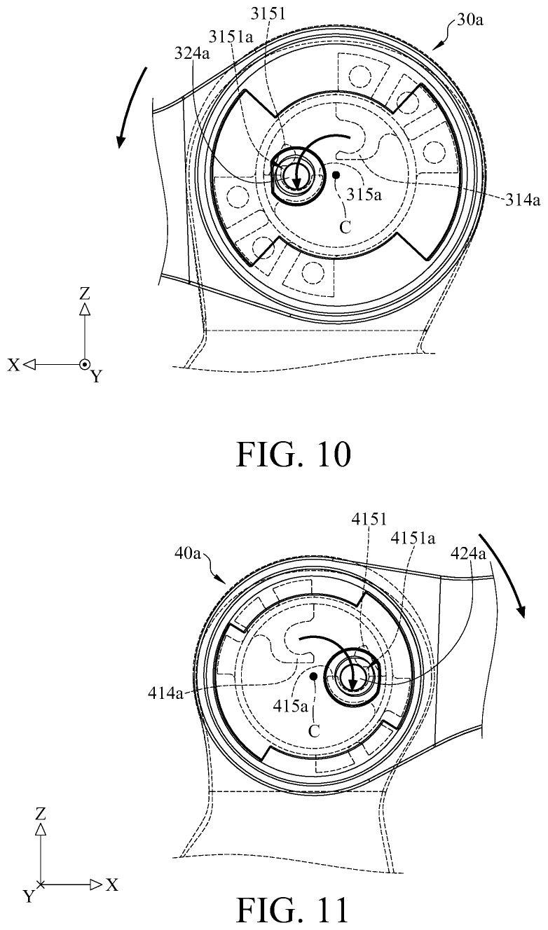

Firstly, please refer to FIG. 9 to FIG. 11; FIG. 9 is a schematic diagram of the ankle foot orthosis 1a as the lower leg piece is folded down, FIG. 10 is a side cross-sectional view of the first pivoting mechanism 30a in FIG. 9, and FIG. 11 is a side cross-sectional view of the second pivoting mechanism 40a in FIG. 9. It is understood that the instep straps 81 and the lower leg straps 82 may be detached before wearing the ankle foot orthosis 1a.

As shown in FIG. 9, the first step is to fold down the lower leg piece 20, and that is to fold down the lower leg piece 20 toward the foot plate 10 about the central pivot axis C, such that the wearer can step his/her foot L down and into the ankle foot orthosis 1a. The first step is labor-saving, easy and convenient for the disabled wearer.

During the first step, the movement of the pin 324a is shown in FIG. 10. In detail, while the lower leg piece 20 is being folded down, the pin 324a of the first pivoting mechanism 30a will be moved toward the second stopper 315a from the position close to the first stopper 314a. During such movement, the pin 324a will press against the free end (not numbered) of the elastic portion 3151 and force the elastic portion 3151 to deform. Then the pin 324a will slide over the free end of the elastic portion 3151 and enter into a first recess 3151a located between the elastic portion 3151 and the second stopper 315a. In this embodiment, as the pin 324a is in the first recess 3151a of the second stopper 315a, the free end of the elastic portion 3151 is able to temporarily hold the pin 324a in the first recess 3151a.

The state of the second pivoting mechanism 40a is similar to that of the first pivoting mechanism 30a. While the lower leg piece 20 is being folded down, the movement of the pin 424a is shown in FIG. 11. In detail, while the lower leg piece 20 is being folded down, the pin 424a of the second pivoting mechanism 40a will be moved toward the fourth stopper 415a from a position closer to the third stopper 414a. During such movement, the pin 424a will press against the free end (not numbered) of the elastic portion 4151 and force the elastic portion 4151 to deform. Then the pin 424a will slide over the free end of the elastic portion 4151 and enter into a second recess 4151a located between the elastic portion 4151 and the fourth stopper 415a. In this embodiment, as the pin 424a is in the second recess 4151a of the fourth stopper 415a, the elastic portion 4151 is able to prevent the pin 424a from being detached from the second recess 4151a.

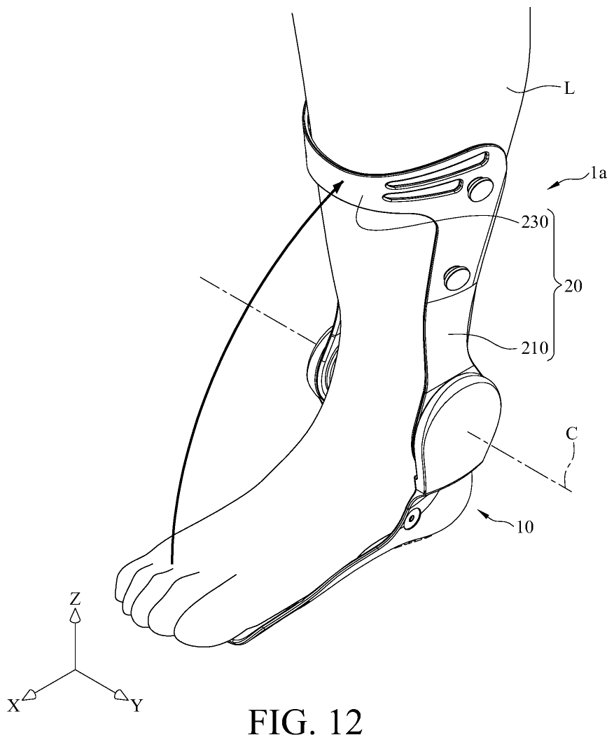

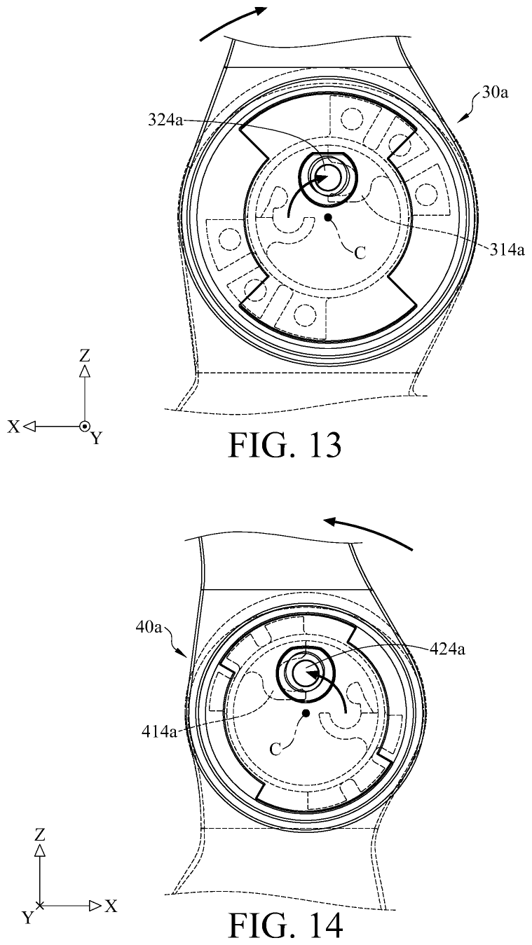

Then, please refer to FIG. 12 to FIG. 14; FIG. 12 is a schematic diagram of the ankle foot orthosis 1a as the lower let piece 20 is folded up, FIG. 13 is a side cross-sectional view of the first pivoting mechanism 30a in FIG. 12, and FIG. 14 is a side cross-sectional view of the second pivoting mechanism 40a in FIG. 12. After the wearer's foot has been in position, the lower leg piece 20 can be folded up to let the bridge part 230 and the upright parts 210 of the lower leg piece 20 to attach to the lower leg, thereby completing positioning the lower leg piece 20.

During this process, as shown in FIG. 13 and FIG. 14, the pin 324a of the first pivoting mechanism 30a is moved toward the first stopper 314a, and the pin 424a of the second pivoting mechanism 40a is moved toward the third stopper 414a.

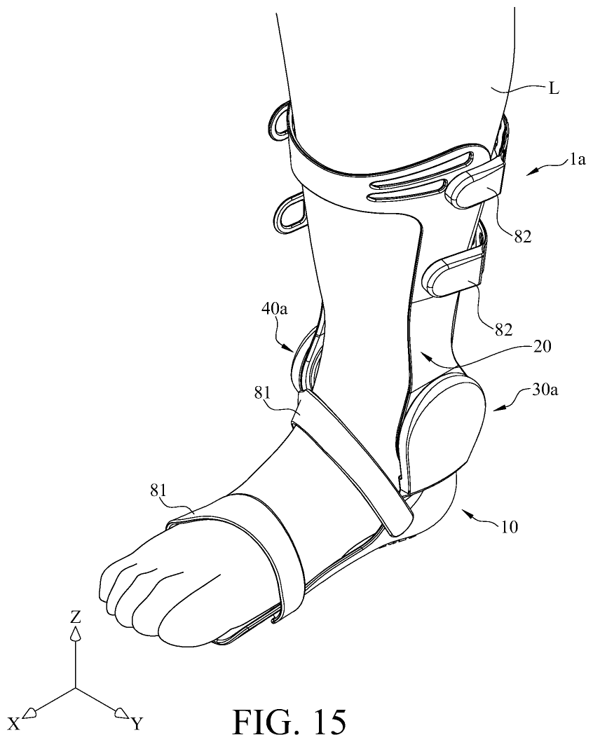

Lastly, please refer to FIG. 15 showing a schematic diagram when an instep strap and a lower leg strap of the ankle foot orthosis according to the first embodiment of the disclosure are fastened in position. As shown in the figure, the last step of wearing the ankle foot orthosis 1a is to fasten the instep straps 81 and lower leg straps 82 to a suitable position.

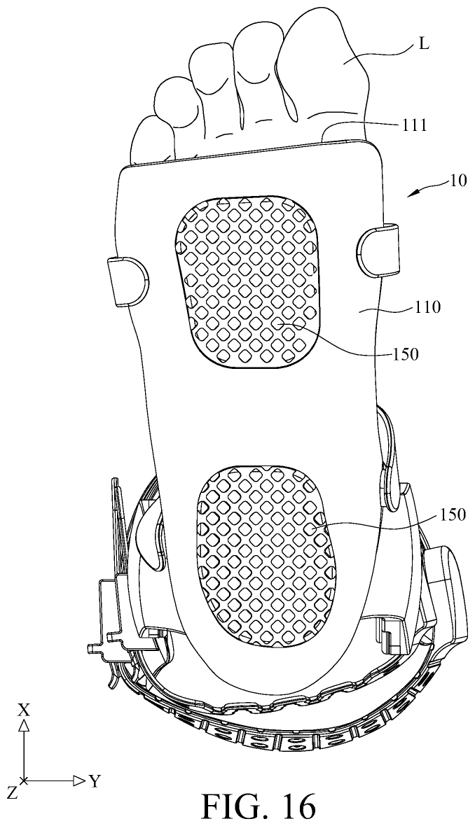

At this moment, as shown in FIG. 16 which is a bottom view of the ankle foot orthosis in FIG. 15. As the ankle foot orthosis 1a is worn, a front edge 111 of the support portion 110 of the foot plate 10 does not cover the wearer's toes in bottom view. That is, the support portion 110 is shorter than the wearer's foot, and the wearer's toes are not blocked by the foot plate 10. This makes the wearer easy to walk while wearing the ankle foot orthosis 1a. Also, as shown in the figure, there may have protruding patterns on the anti-slip pads 150 to improve the friction from the ground, but the disclosure is no limited by the patterns on the anti-slip pads 150.

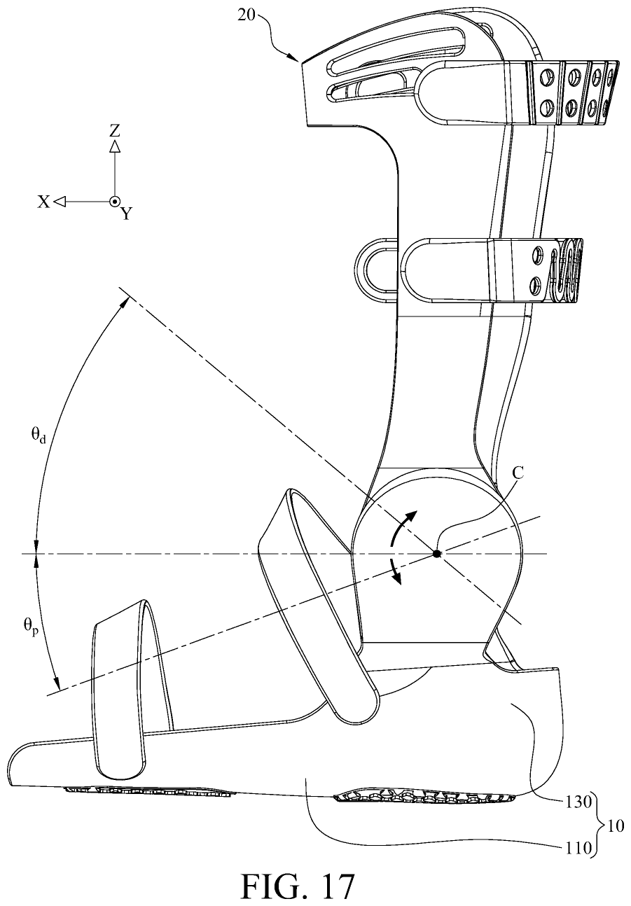

Furthermore, due to the designs of the first pivoting mechanism 30a and the second pivoting mechanism 40a, the motion of the ankle joint is limited to a specific range. Please refer to FIG. 17 to see a schematic diagram showing the usage of the ankle foot orthosis 1a in FIG. 15. As shown in the figure, in this embodiment, the first pivoting mechanism 30a and the second pivoting mechanism 40a only allow a limited amount of pivotal movement of the foot plate 10 with respect to the lower leg piece 20 such as about 60 degrees. In detail, if the state when the footplate 10 is approximately perpendicular to the lower leg piece 20 is defined as a beginning state, the first pivoting mechanism 30a and the second pivoting mechanism 40a can limit the foot plate 10 to perform about 20-degree of the maximum plantar flexion (i.e., the maximum plantar flexion angle θₚ) and limit the foot plate 10 to perform about 40-degree of the maximum dorsiflexion (i.e., the maximum dorsiflexion angle θ_{d}).

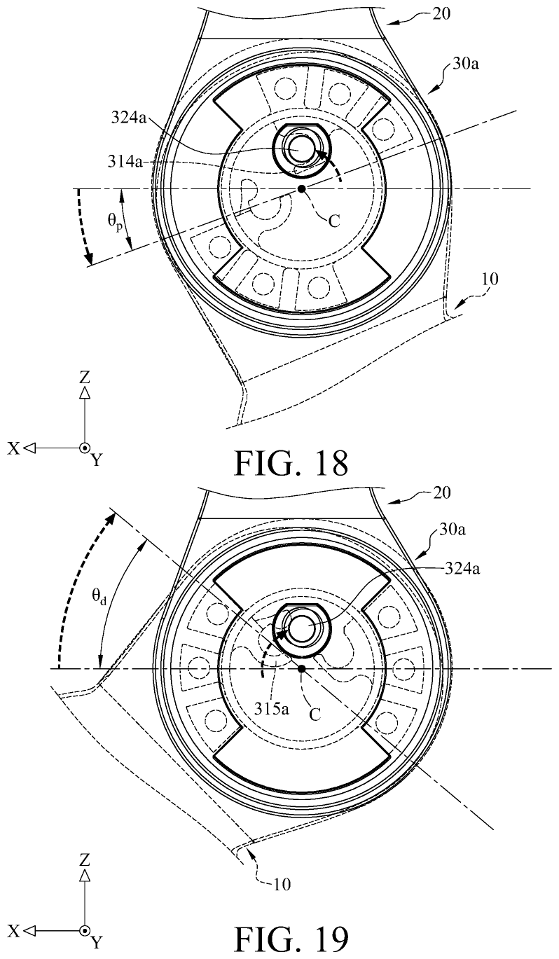

Take the first pivoting mechanism 30a for example. Please see FIG. 18 to see a side cross-sectional view of the first pivoting mechanism 30a in FIG. 15 during plantar flexion. When the wearer performs the plantar flexion exercise from the beginning state so as to swing the foot plate 10 downward relatively to the lower leg piece 20 (in the figure, the foot plate 10 is moved in counterclockwise direction), the first stopper 314a is moved toward and engaged with the pin 324a (i.e., the pin 324a is moved toward and engaged with the first stopper 314a). Therefore, the maximum angle of swing downward the foot plate 10 from the beginning state is only about 20 degrees; that is, the permitted angle of the maximum plantar flexion exercise from the beginning state is limited at about 20 degrees.

On the other hand, please see FIG. 19 to see a side cross-sectional view of the first pivoting mechanism 30a in FIG. 15 during dorsiflexion. When the wearer performs the dorsiflexion exercise from the beginning state so as to swing the foot plate 10 upward relatively to the lower leg piece 20 (in the figure, the foot plate 10 is moved in clockwise direction), the second stopper 315a is moved toward and engaged with the pin 324a (i.e., the pin 324a is moved toward and engaged with the second stopper 315a). Therefore, the maximum angle of swinging upward the foot plate 10 from the beginning state is only about 40 degrees; that is, the permitted angle of the maximum dorsiflexion exercise from the beginning state is limited at about 40 degrees.

As discussed above, in the first pivoting mechanism 30a, the pivotal movement of the second connecting part 320a would be stopped as the pin 324a is stopped by the first stopper 314a or the second stopper 315a. That is, the movement range of the pin 324a between the first stopper 314a and the second stopper 315a determines the amount of the pivotable movements of the first connecting part 310a and the second connecting part 320a. Therefore, it is understood that the angle between the first stopper 314a and second stopper 315a with respect to the central pivot axis C determines the amount of the pivotable movement of the first connecting part 310a and the second connecting part 320a. As such, when the first connecting part 310a is pivoted relatively to the second connecting part 320a, the pin 324a will be stopped by the first stopper 314a or the second stopper 315a at a specific position. In this embodiment, the angle of the inner area between the first stopper 314a and the second stopper 315a with respect to the central pivot axis C is about 0 to 90 degrees, such that the pivotable movement of the first connecting part 310a relative to the second connecting part 320a is limited.

Similarly, in the second pivoting mechanism 40a, the pivotal movement of the fourth connecting part 420a would be stopped as the pin 424a is stopped by the third stopper 414a or the fourth stopper 415a. That is, the movement range of the pin 424a between the third stopper 414a and the fourth stopper 415a determines the amount of the pivotable movement of the third connecting part 410a and the fourth connecting part 420a. Therefore, it is understood that the angle between the third stopper 414a and fourth stopper 415a with respect to the central pivot axis C determines the pivotable movement of the third connecting part 410a and the fourth connecting part 420a. As such, when the third connecting part 410a is pivoted relatively to the fourth connecting part 420a, the pin 424a will be stopped by the third stopper 414a or the fourth stopper 415a at a specific position. The angle of the inner area between the fourth stopper 415a and third stopper 414a with respect to the central pivot axis C is about or larger than 90. Since the second pivoting mechanism 40a is similar to the first pivoting mechanism 30a, the respective drawings are omitted. It is noted that the disclosure is not limited by the above value or range of angles, the actual locations among the above stoppers may be changed according to the requirement of the wearer. That is to say, in other embodiments, the ankle foot orthosis may allow the wearer to perform a larger angle of plantar flexion and dorsiflexion exercises based on actual requirements.

Moreover, to make the wearer easily figure out the ankle foot orthosis 1a is for left or right foot, the first pivoting mechanism 30a and the second pivoting mechanism 40a are not symmetric in appearance. In detail, in this or other embodiments, projections of the first pivoting mechanism 30a and the second pivoting mechanism 40a on an imaginary plane (not shown) perpendicular to the central pivot axis C have different sizes. Briefly, from the view along the central pivot axis C, the first pivoting mechanism 30a and the second pivoting mechanism 40a have different sizes of side view. As such, the wearer may visually determine whether the ankle foot orthosis 1a is for the left or right foot, achieving fool-proofing. However, the disclosure is not limited thereto. In other embodiments, the projections of the first pivoting mechanism and the second pivoting mechanism onto the imaginary plane perpendicular to the central pivot axis may substantially have the same size. That is, the first pivoting mechanism and the second pivoting mechanism may be similar or the same in appearance.

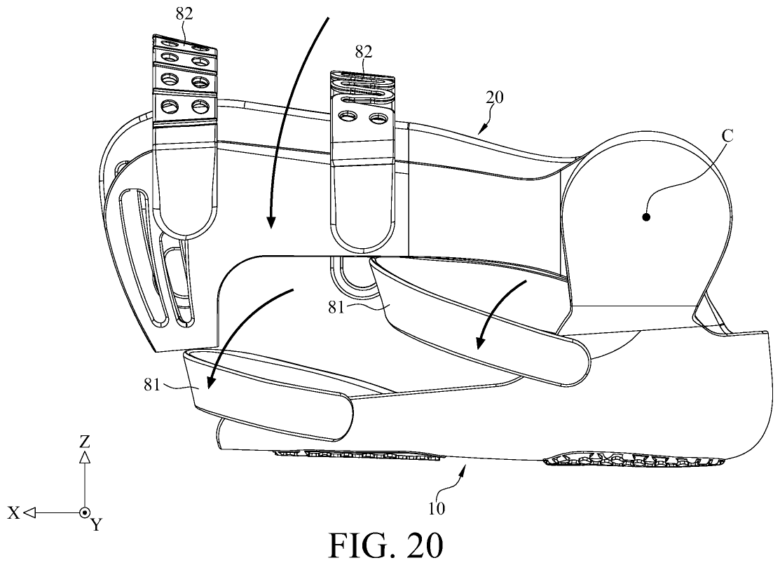

It is understood that, the wearer may take off the ankle foot orthosis 1a by reversing the above steps shown in FIG. 9, FIG. 12 and FIG. 15, so it will not be described in detail. In addition, according to the state of the ankle foot orthosis 1a in FIG. 9, the ankle foot orthosis 1a is able to be stored in a small space and becomes very portable. As shown in FIG. 20, the instep straps 81 may further be pulled down to fold the ankle foot orthosis 1a and the lower leg piece 20, so that the ankle foot orthosis 1a is able to be stored in a small space and becomes very portable.

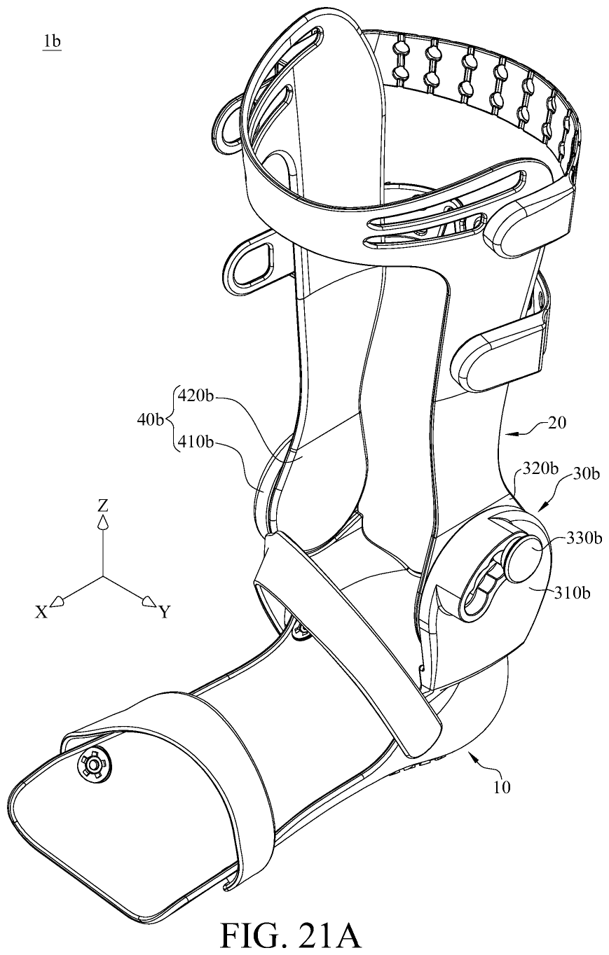

The above descriptions are related to the ankle foot orthosis 1a of the first embodiment, but the disclosure is not limited thereto. Please refer to, FIG. 21A to FIG. 21B; FIG. 21A to FIG. 21B respectively are perspective views of an ankle foot orthosis 1b according to a second embodiment of the disclosure at different viewing angles.

The disclosure provides another embodiment of ankle foot orthosis 1b that is also pivotable. The main differences between the ankle foot orthosis 1b and the ankle foot orthosis la are the joints. Therefore, the following paragraphs are focus on the introduction of the joints of the ankle foot orthosis 1b, and the similar parts of these two embodiments are omitted.

In this embodiment, the ankle foot orthosis 1b includes the aforementioned foot plate 10, the aforementioned lower leg piece 20, a first pivoting mechanism 30b, a second pivoting mechanism 40b, the aforementioned instep straps 81, and the aforementioned lower leg straps 82.

The foot plate 10 and the lower leg piece 20 may be pivotably connected to each other via the first pivoting mechanism 30b and the second pivoting mechanism 40b so that the foot plate 10 and the lower leg piece 20 are allowed to be pivoted relatively to each other along the central pivot axis C, thereby allowing the wearer's leg to perform plantar flexion or dorsiflexion exercise.

The first pivoting mechanism 30b includes a first connecting part 310b, a second connecting part 320b, and a pin 330b. The second pivoting mechanism 40b includes a third connecting part 410b and a fourth connecting part 420b. The connections and relationships among the first connecting part 310b and the second connecting part 320b of the first pivoting mechanism 30b, the third connecting part 410b and the fourth connecting part 420b of the second pivoting mechanism 40b, and the foot plate 10 and the lower leg piece 20 are similar to that of the previous embodiments and thus will not be repeated hereinafter.

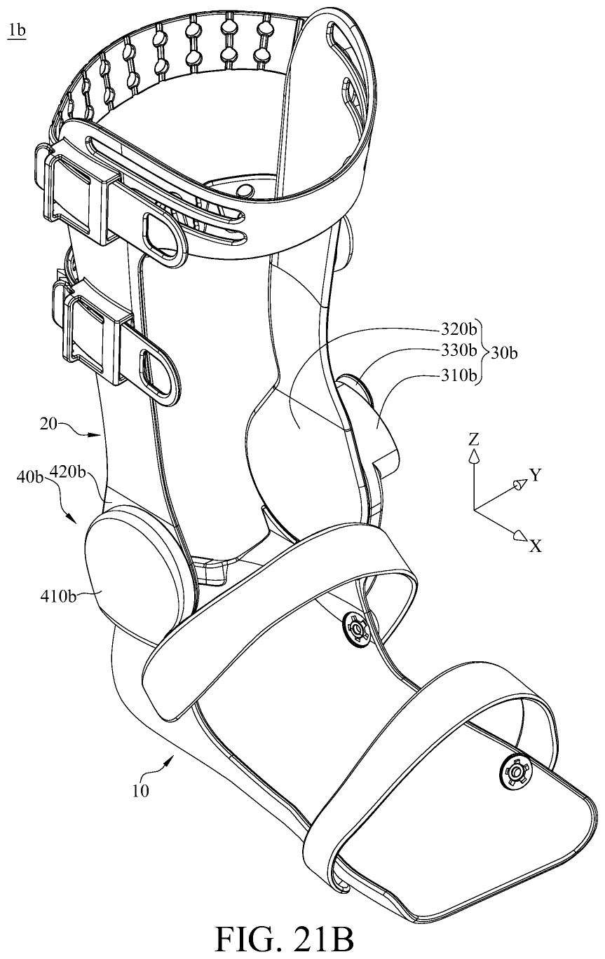

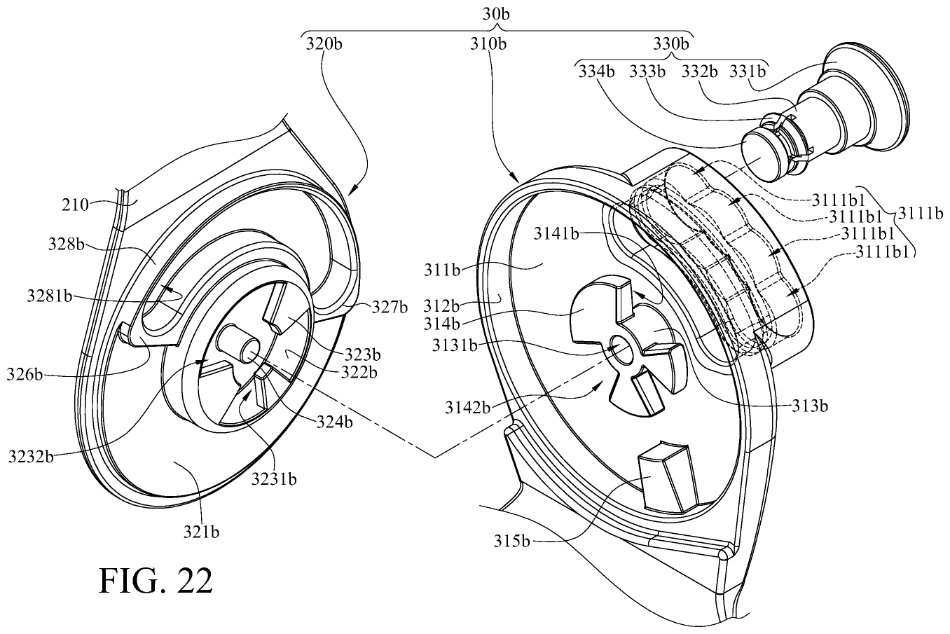

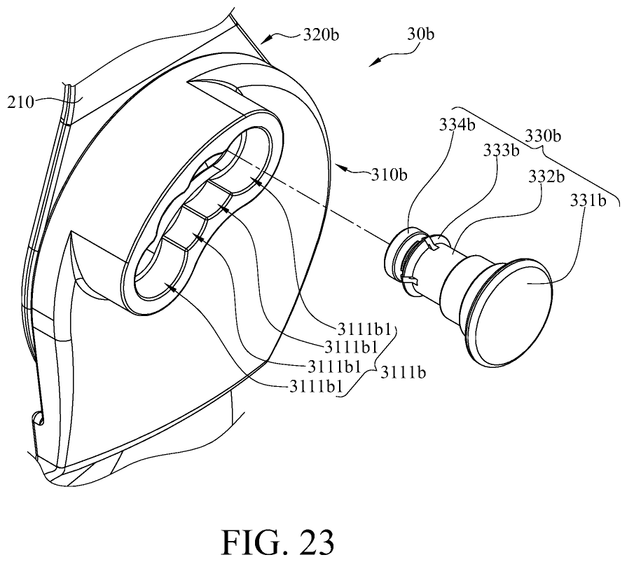

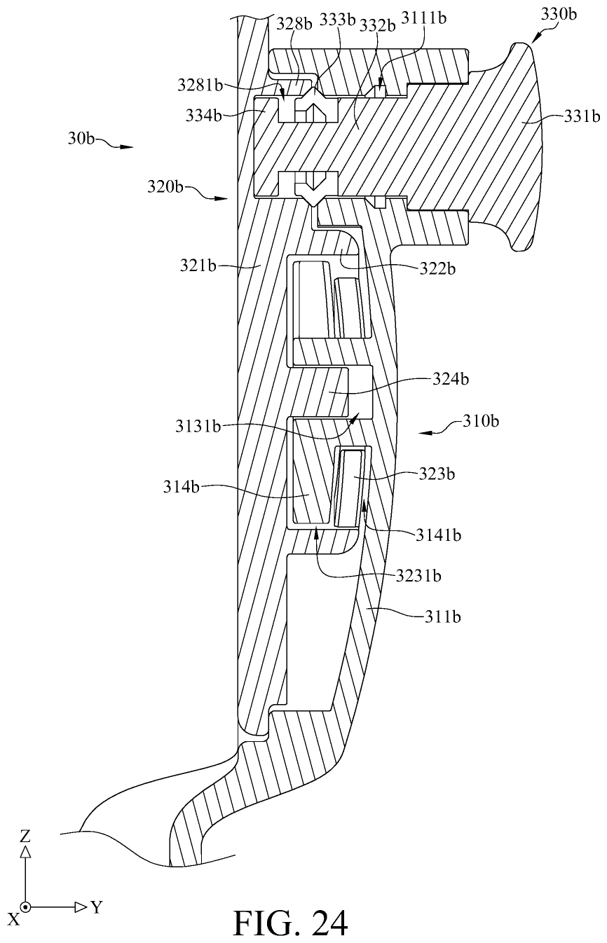

The first pivoting mechanism 30b and the second pivoting mechanism 40b are then introduced in detail. Firstly, the first pivoting mechanism 30b is introduced. Please refer to FIGS. 22 to 24; FIG. 22 is an exploded view of the first pivoting mechanism 30b of the ankle foot orthosis 1b, FIG. 23 is a partially enlarged exploded view of the first pivoting mechanism 30b in FIG. 21A, and FIG. 24 is a partial side cross-sectional view of the first pivoting mechanism 30b in FIG. 21A.

In this embodiment, the second connecting part 320b of the first pivoting mechanism 30b is pivotably disposed on the inner side of the first connecting part 310b of the first pivoting mechanism 30b. The first connecting part 310b of the first pivoting mechanism 30b includes a first plate portion 311b, a first sidewall portion 312b, a first pillar 313b, a plurality of first engagement portions 314b, and a block 315b.

The first plate portion 311b is connected to one side of the support portion 110, and the first sidewall portion 312b protrudes toward the second connecting part 320b form the first plate portion 311b. The first pillar 313b protrudes from the surface of the first plate portion 311b facing the second connecting part 320b, and the first pillar 313b has a first pivot hole 3131b whose axis (not shown) substantially overlaps the central pivot axis C. The first engagement portions 314b extend outward from a side of the first pillar 313b away from the first plate portion 311b along radial directions. The first engagement portions 314b are spaced apart from the first plate portion 311b so that a first groove 3141b is formed therebetween. The first engagement portions 314b are roughly arc-shaped and are spaced apart from each other in arc-shaped direction so that a plurality of openings 3142b are formed between the first engagement portions 314b in arc-shaped direction. The openings 3142b are roughly arc-shaped. The block 315b protrudes from the surface of the first plate portion 311b facing the second connecting part 320b and is disposed near the first sidewall portion 312b. In addition, the first plate portion 311b has an insertion hole 3111b which is a through hole on the first plate portion 311b and is disposed near the first sidewall portion 312b for the insertion of the pin 330b. Further, the insertion hole 3111b is formed by a plurality of sub insertion holes 3111b1. The sub insertion holes 3111b1 are connected to one another and arranged along an arc-shaped direction, and each sub insertion hole 3111b1 is configured for the insertion of the pin 330b. However, the configuration of the sub insertion holes 3111b1 are not restricted and the quantity of the sub insertion holes 3111b1 may be changed according to the actual requirements. In addition, to the insertion hole 3111b, the angle between two opposite ends of the insertion hole 3111b with respect to the central pivot axis C ranges approximately 165 to 225 degrees.

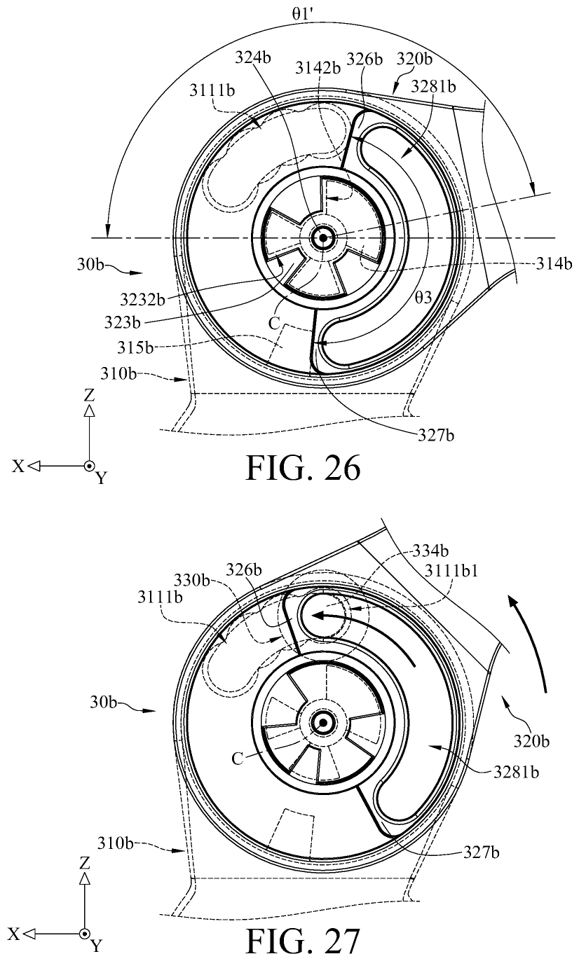

The second connecting part 320b of the first pivoting mechanism 30b includes a second plate portion 321b, a second sidewall portion 322b, a plurality of second engagement portions 323b, a first pivot pillar 324b, a first stopper 326b, a second stopper 327b, and an arc-shaped sidewall 328b. The second plate portion 321b is connected to one of the upright parts 210. The second sidewall portion 322b protrudes toward the first connecting part 310b from the second plate portion 321b. The second engagement portions 323b are respectively located on different sides of the second sidewall portion 322b and extend inward from a side of the second sidewall portion 322b facing away from the second plate portion 321b along radial directions. The second engagement portions 323b are spaced apart from the second plate portion 321b so that a second groove 3231b is formed therebetween. The second engagement portions 323b are roughly arc-shaped and are spaced apart from each other in arc-shaped direction so that two openings 3232b are formed between the second engagement portions 323b. The openings 3232b are roughly arc-shaped and directly connected to the second groove 3231b. The first pivot pillar 324b protrudes from the surface of the second plate portion 321b facing the first connecting part 310b, and its axis (not shown) substantially overlaps the central pivot axis C so that the first pivot pillar 324b corresponds to the first pivot hole 3131b of the first pillar 313b. The first stopper 326b and the second stopper 327b extend outward from the outer surface of the second sidewall portion 322b along radial directions. The arc-shaped sidewall 328b connects the first stopper 326b and the second stopper 327b so as to form a guide groove 3281 with the outer surfaces of the first stopper 326b, second stopper 327b and second sidewall portion 322b. In this embodiment, the first stopper 326b and the second stopper 327b have an angle θ3 (as shown in following FIG. 26) with respect to the central pivot axis C. The angle θ3 ranges about 170 to 180 degrees, but the disclosure is not limited thereto. In this embodiment, the angle θ3 may be, for example, 175 degrees. In other embodiments, the angle θ3 may be adjusted to be any angle between 165 and 225 degrees.

The pin 330b of the first pivoting mechanism 30b is also detachable. The pin 330b includes a head part 331b, a neck part 332b, a ring-shaped protrusion 333b, and an end part 334b. The neck part 332b is connected to and located between the head part 331b and the ring-shaped protrusion 333b, the diameter of the neck part 332b is between that of the head part 331b and that of the ring-shaped protrusion 333b, and the ring-shaped protrusion 333b is connected to and located between the neck part 332b and the end part 334b. The head part 331b and the end part 334b are understood to be respectively two opposite ends of the pin 330b. The ring-shaped protrusion 333b is elastic so that it can be deformed and recover from the deformation.

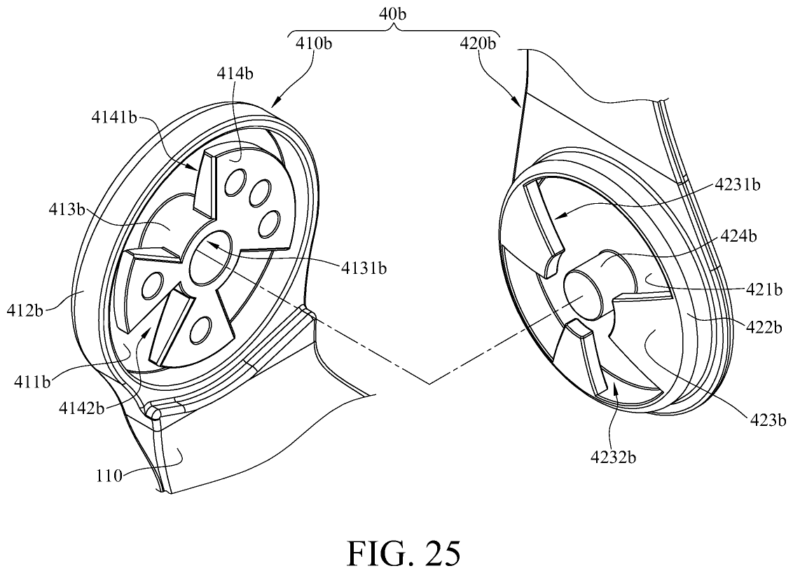

Then, please refer to FIG. 25 to see an exploded view of the second pivoting mechanism 4b. In this embodiment, the fourth connecting part 420b of the second pivoting mechanism 40b is pivotably disposed on the inner side of the third connecting part 410b of the second pivoting mechanism 40b. The third connecting part 410b of the second pivoting mechanism 40b includes a third plate portion 411b, a third sidewall portion 412b, a second pillar 413b, and a plurality of third engagement portions 414b. The third plate portion 411b is connected to another side of the support portion 110, and the third sidewall portion 412b protrudes toward the fourth connecting part 420b from the third plate portion 411b. The second pillar 413b protrudes from the surface of the third plate portion 411b facing the fourth connecting part 420b, and the second pillar 413b has a second pivot hole 4131b whose axis (not shown) substantially overlaps the central pivot axis C. The third engagement portions 414b extend outward from a side of the second pillar 413b away from the third plate portion 411b along radial direction. The third engagement portions 414b are spaced apart from the third plate portion 411b to form a third groove 4141b therebetween. The third engagement portions 414b are roughly arc-shaped and spaced apart from each other in arc-shaped direction so that a plurality of openings 4142b are formed between the third engagement portions 414b along arc-shaped direction.

The fourth connecting part 420b of the second pivoting mechanism 40b includes a fourth plate portion 421b, a fourth sidewall portion 422b, a plurality of fourth engagement portions 423b, and a second pivot pillar 424b. The fourth plate portion 421b is connected to one of the upright parts 210. The fourth sidewall portion 422b protrudes toward the third connecting part 410b from the fourth plate portion 421b. The fourth engagement portions 423b are respectively located on different sides of the fourth sidewall portion 422b, and extend inward from a side of the fourth sidewall portion 422b facing away from the fourth plate portion 421b along radial directions. The fourth engagement portions 423b are spaced apart from the fourth plate portion 421b so that a fourth groove 4231b is formed therebetween. The fourth engagement portions 423b are roughly arc-shaped and are spaced apart from each other in arc-shaped direction so that a plurality of openings 4232b are formed between the fourth engagement portions 423b along arc-shaped direction. The openings 4232b are roughly arc-shaped and are the structures that form and are directly connected to the aforementioned fourth groove 4231b. The second pivot pillar 424b protrudes from the surface of the fourth plate portion 421b facing the third connecting part 410b, and its axis (not shown) substantially overlaps the central pivot axis C so that the second pivot pillar 424b corresponds the second pivot hole 4131b of the second pillar 413b.

Then, the way to assemble the first pivoting mechanism 30b and the second pivoting mechanism 40b will be introduced. Please refer to FIG. 26 to FIG. 27; FIG. 26 is a side cross-sectional view of the first pivoting mechanism 30b when the first pivoting mechanism 30 is in an assemblable state, and FIG. 27 is a schematic assembly diagram showing the pin in the first pivoting mechanism 30b. To assemble the first pivoting mechanism 30b, the first engagement portions 314b of the first connecting part 310b may be pivoted to the openings 3232b of the second connecting part 320b, and which is to simultaneously pivot the second engagement portions 323b of the second connecting part 320b to the openings 3142b of the first connecting part 310b. During this process, the second stopper 327b of the second connecting part 320b will press against and be stopped by the block 315b of the first connecting part 310b, thereby noticing the wearer that the first pivoting mechanism 30b is in position. In this moment, the first pivoting mechanism 30b is in an assemblable state (i.e., the detachable state) thereof, and the first engagement portions 314b are allowed to be placed into the openings 3232b, meanwhile, the second engagement portions 323b are allowed to be placed into the openings 3142b, such that the first pivot pillar 324b of the second connecting part 320b can be inserted into the first pivot hole 3131b of the first connecting part 310b. When the first pivoting mechanism 30b is in the assemblable state (or detachable position) thereof, the insertion hole 3111b on the first connecting part 310b for the insertion of the pin 330b is entirely located above the outer area of the first stopper 326b and the second stopper 327b of the second connecting part 320b, and that is to say that the insertion hole 3111b is not located above the guide groove 3281b and does not overlap with the guide groove 3281b. In addition, in this or other embodiments, the area between the first stopper 326b and the second stopper 327b and available for the movement of the pin 330b as the pin 330b is assembled in position is defined as the inner area of the first stopper 326b and the second stopper 327b, the other area is an outer area of the first stopper 326b and the second stopper 327b.

Then, the first connecting part 310b and the second connecting part 320b may be pivoted relatively to each other so that the first engagement portions 314b is engaged into the second groove 3231b of the second connecting part 320b (as indicated in FIG. 25), and that is to simultaneously engage the second engagement portions 323b into the first groove 3141b of the first connecting part 310b. As such, the first connecting part 310b and the second connecting part 320b are engaged with each other to make the first pivoting mechanism 30b in an engaged state. As shown in FIG. 27, the first connecting part 310b and the second connecting part 320b may be pivoted relatively to each other to be moved to a position of at least one of the sub insertion holes 3111b1 of the insertion hole 3111b between the first stopper 326b and the second stopper 327b of the second connecting part 320b, and that is to move the insertion hole 3111b to be located above the guide groove 3281b to partially overlap with the guide groove 3281b. In this moment, the pin 330b may be selectively inserted into one of the sub insertion holes 3111b1 of the insertion hole 3111b so as to protrude from the surface of the first connecting part 310b facing the second connecting part 320b, such that one end thereof (i.e. the end part 334b) will be in the guide groove 3281b. During the insertion, the ring-shaped protrusion 333b of the pin 330b will be squeezed and deformed while it goes through the insertion hole 3111b, but after the ring-shaped protrusion 333b moves out of the insertion hole 3111b, the ring-shaped protrusion 333b will recover from the deformation and fix the pin 330b to the first connecting part 310b. By doing so, assembling the first pivoting mechanism 30b is completed. At this moment, the end part 334b of the pin 330b is located in the guide groove 3281b and located between the first stopper 326b and the second stopper 327b. The relationship among the pin 330b with respect to the first stopper 326b and the second stopper 327b during the motion of the ankle will be described later.

It is understood that it is easy to disassemble the first pivoting mechanism 30b by reversing the above steps during the assembly. In concrete, first is to remove the pin 330b or pull it out to a position that will not interfere with the first stopper 326b and the second stopper 327b, second is to pivot the first connecting part 310b and the second connecting part 320b to the state shown in FIG. 26 to allow the first engagement portions 314b to be removed from the openings 3232b (i.e., to remove the first engagement portions 314b from the second groove 3231b) and to allow the second engagement portions 323b to be removed from the openings 3142b (i.e., to remove the second engagement portions 323b from the first groove 3141b), and the last is to detach the lower leg piece 20 and the foot plate 10 from the first pivoting mechanism 30b.

Then, please refer to FIG. 28 to see a side cross-sectional view of the second pivoting mechanism when the second pivoting mechanism is in an assemblable state. To assemble the second pivoting mechanism 40b, firstly, the third engagement portions 414b of the third connecting part 410b may be pivoted to the position of the openings 4232b of the fourth connecting part 420b, and that is to simultaneously pivot the fourth engagement portions 423b of the fourth connecting part 420b to the position of the openings 4142b of the third connecting part 410b. In this moment, the second pivoting mechanism 40b is in an assemblable state (i.e., detachable state) thereof, and that is to say that the third engagement portions 414b are allowed to be placed into the openings 4232b, meanwhile, the fourth engagement portions 423b are allowed to be placed into the openings 4142b, such that the third sidewall portion 412b of the third connecting part 410b will cover the fourth sidewall portion 422b of the fourth connecting part 420b. Then, the third connecting part 410b and the fourth connecting part 420b may be pivoted relatively to each other so that the third engagement portions 414b will be engaged into the fourth groove 4231b of the fourth connecting part 420b (as indicated in FIG. 25) and the fourth engagement portions 423b will be engaged into the third groove 4141b of the third connecting part 410b. As a result, the third connecting part 410b and the fourth connecting part 420b are engaged with each other to make the second pivoting mechanism 40b in the engaged state.

In contrary, to disassemble the second pivoting mechanism 40b, first is to pivot the third connecting part 410b or the fourth connecting part 420b to move the third engagement portions 414b to the openings 4232b of the fourth connecting part 420b (i.e., to move the fourth engagement portions 423b out of the fourth groove 4231b) and to move the fourth engagement portions 423b to the openings 4142b of the third connecting part 410b (i.e., to move the fourth engagement portions 423b out of the third groove 4141b). By doing so, the lower leg piece 20 and the foot plate 10 can be detached from each other from the second pivoting mechanism 40b.

Additionally, it is explained that the assemble/disassemble angles of the first pivoting mechanism 30b and the second pivoting mechanism 40b are different. In detail, please compare the FIG. 26 and FIG. 28, as shown in FIG. 26, when the first pivoting mechanism 30b is in the assemblable state (i.e., detachable state) thereof, an extension direction of the lower leg piece 20 passing through the central pivot axis C has a first angle θ1′ at imaginary line that passes through the central pivot axis C and is parallel to the ground. The first angle θ1′ may be considered as the angle between the foot plate 10 and the lower leg piece 20. When the second pivoting mechanism 40b is in the assemblable state (i.e., detachable state) as shown in FIG. 28, the extension direction of the lower leg piece 20 passing through the central pivot axis C has a second angle θ2′ at an imaginary line which passes through the central pivot axis C and is parallel to the ground. The second angle θ2′ may be considered as the angle between the foot plate 10 and the lower leg piece 20, but the second angle θ2′ is different from the said first angle θ1′. That is, the foot plate 10 and the lower leg piece 20 should be pivoted relatively to each other to different angles to assemble the first pivoting mechanism 30b and the second pivoting mechanism 40b respectively. This helps the user to assemble the first pivoting mechanism 30b and the second pivoting mechanism 40b one at a time, thereby preventing the inconvenience for assembling two opposite adjustment mechanisms. The way to disassemble the first pivoting mechanism 30b and the second pivoting mechanism 40b is similar to the way to assemble the same. This helps the user to disassemble the first pivoting mechanism 30b and the second pivoting mechanism 40b one at a time, avoiding the first pivoting mechanism 30b and the second pivoting mechanism 40b from falling apart at the same time during the disassembly process.

Then, the following will explain how to wear the ankle foot orthosis 1b and further explain the relationships among the internal configurations of the first pivoting mechanism 30b and the second pivoting mechanism 40b.

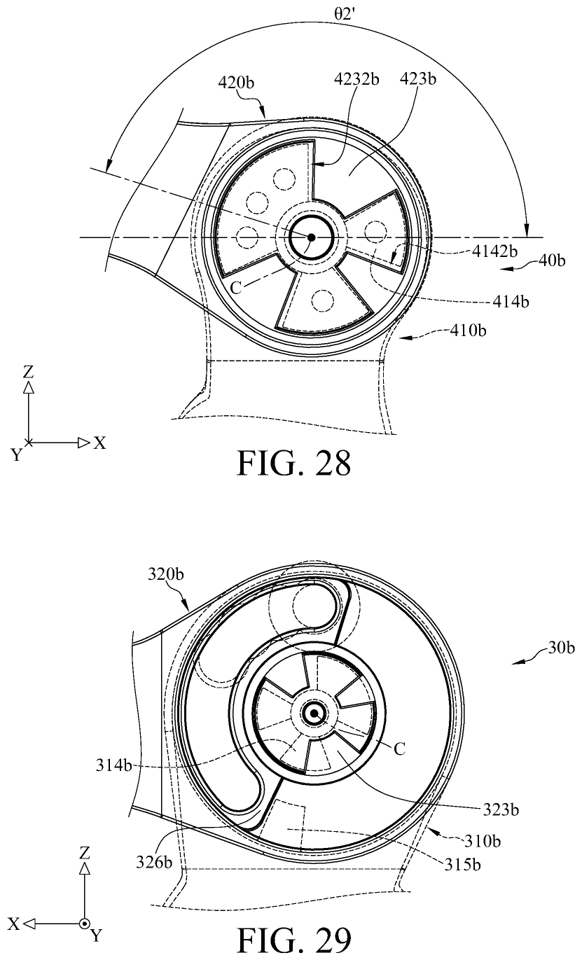

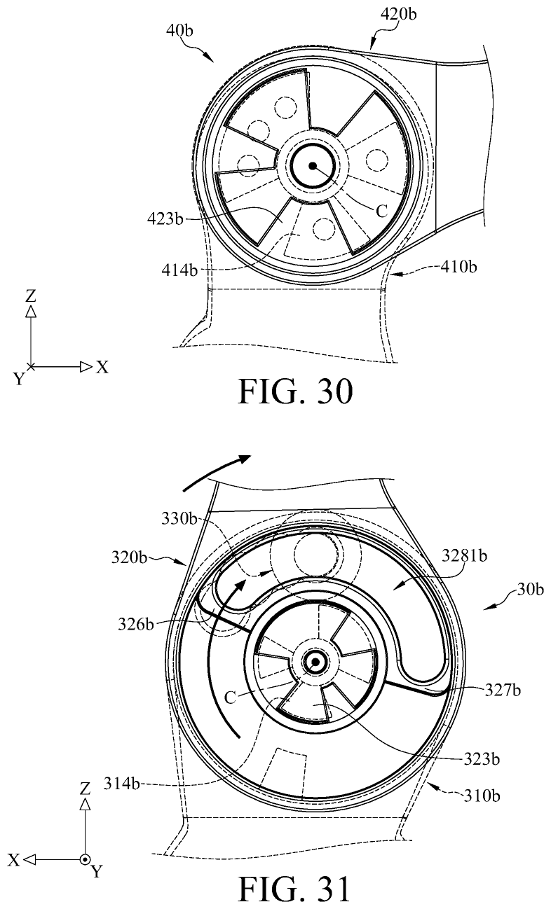

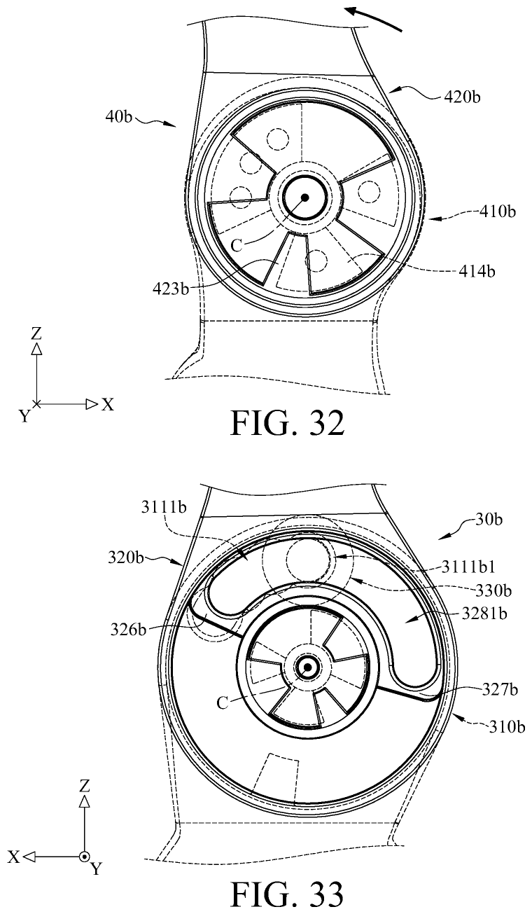

Firstly, please refer to FIG. 29 to FIG. 32; FIG. 29 to FIG. 30 are side cross-sectional views of the first pivoting mechanism and the second pivoting mechanism when the lower leg piece is pivoted and folded down on the foot plate, and FIG. 31 to FIG. 32 are side cross-sectional views of the first pivoting mechanism and the second pivoting mechanism when the lower leg piece is folded up to be attached to the lower leg.

In general, the steps regarding how to wear the ankle foot orthosis 1b can be referred to the previous embodiments, except for the insertion of the pin 330b is different and required to be further explained.

In detail, the first step of wearing the ankle foot orthosis 1b may be referred to that of the first embodiment in FIG. 9; the lower leg piece 20 may be folded down, but the related drawings are omitted since they are similar in appearance. In this moment, the internal states of the first pivoting mechanism 30b and the second pivoting mechanism 40b are respectively shown in FIG. 29 and FIG. 30. In FIG. 29, while the lower leg piece 20 is being folded down, the first stopper 326b of the second connecting part 320b of the first pivoting mechanism 30b will press against the block 315b of the first connecting part 310b of the first pivoting mechanism 30b so as to maintain the lower leg piece 20 in the current folded position with respect to the foot plate 10, and the first engagement portions 314b and the second engagement portions 323b of the first pivoting mechanism 30b are engaged with each other. In FIG. 30, the third engagement portions 414b and the fourth engagement portions 423b of the second pivoting mechanism 40b are maintained in the engaged state.

Then, the second step is to fold the lower leg piece 20 up, and which can be referred to the respective content in the first embodiment in FIG. 12 and thus the related drawings are omitted since they are similar in appearance. In this moment, as shown in FIG. 31, when the lower leg piece 20 is pivoted, the pin 330b will be slid relatively to the guide groove 3281b of the first pivoting mechanism 30b, and the first engagement portions 314b and the second engagement portions 323b of the first pivoting mechanism 30b are still engaged with each other; as shown in FIG. 32, the third engagement portions 414b and the fourth engagement portions 423b of the second pivoting mechanism 40b are also engaged with each other.

Lastly, the instep straps 81 and lower leg straps 82 are fastened to a suitable position to complete wearing the ankle foot orthosis 1b as shown in the first embodiment in FIG. 15, but the related drawings are omitted since they are similar in appearance.

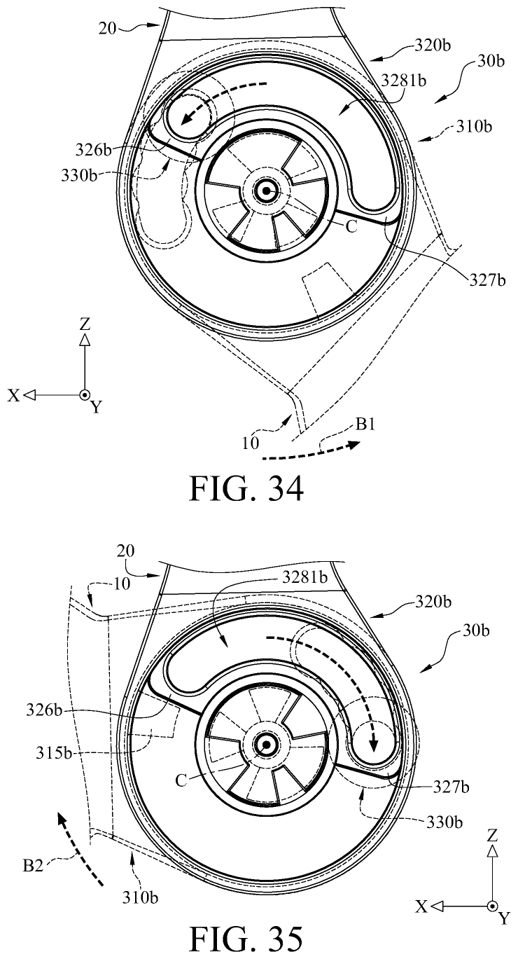

Similarly, the ankle foot orthosis 1b is also able to restrict the motion of the ankle within a specific range. For example, please refer to FIG. 33 to FIG. 35 to see schematic diagrams showing the movement of the pin of the first pivoting mechanism in one of the insertion positions. First, for example, the pin 330b is inserted into the rightmost sub insertion hole 3111b1 of the insertion hole 3111b. At this moment, the foot plate 10 is allowed to be pivoted relatively to the lower leg piece 20 along a direction indicated by arrow B1 (counterclockwise direction in the drawings) as shown in FIG. 33 and FIG. 34, such that the foot plate 10 can be pivoted from the position in FIG. 33 to that in FIG. 34 so as to allow the wearer to perform a certain amount of plantar flexion exercise until the pin 330b is stopped by the first stopper 326b; or, as shown in FIG. 33 and FIG. 35, the foot plate 10 may be pivoted relatively to the lower leg piece 20 along a direction indicated by arrow B2 (clockwise direction in the drawings), such that the foot plate 10 can be pivoted from the position in FIG. 33 to that in FIG. 35 so as to allow the wearer to perform a certain amount of dorsiflexion exercise until the pin 330b is stopped by the second stopper 327b.

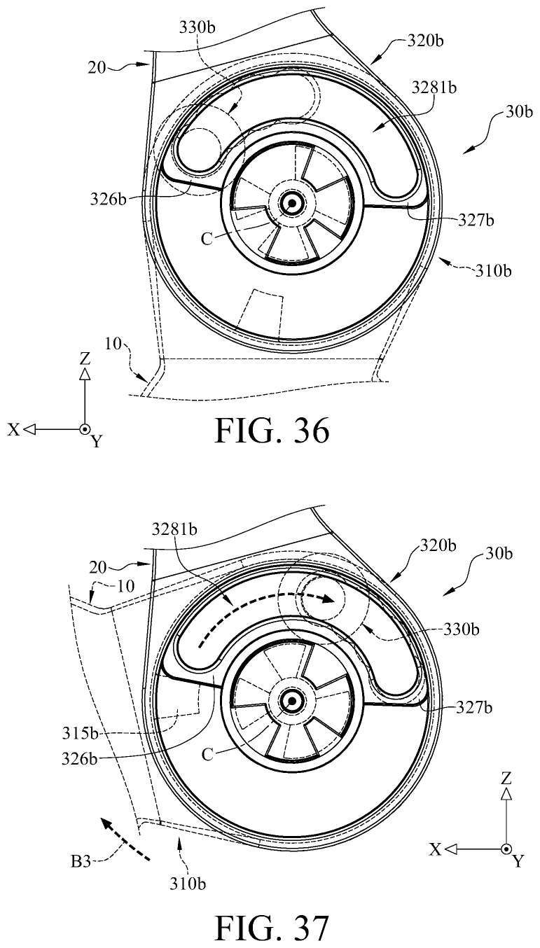

Alternatively, please refer to FIG. 36 to FIG. 37, when the pin 330b is inserted into the leftmost sub insertion hole 3111b1 of the insertion hole 3111b, the foot plate 10 may be pivoted relatively to the lower leg piece 20 along a direction indicated by arrow B3 (counterclockwise direction in the drawings), such that the foot plate 10 can be moved from the position in FIG. 36 to that in FIG. 39 so as to allow the wearer to perform a larger amount of dorsiflexion exercise. However, in this case, the initial position of the pin 330b is nearly in contact with the first stopper 326b so that the foot plate 10 is indirectly limited and cannot be pivoted relatively to the lower leg piece 20 along the direction opposite to arrow B3; that is, the plantar flexion is not allowed in such a position.

As discussed above, while the second connecting part 320b is pivoted relatively to the first connecting part 310b, the first connecting part 310b will be stopped by the first stopper 326b or the second stopper 327b. In other words, when first connecting part 310b is pivoted relatively to the second connecting part 320b, the first connecting part 310b will be stopped by the first stopper 326b or the second stopper 327b at a specific position so that the pivotable movement of the foot plate 10 with respect to the lower leg piece 20 is restricted to a specific range.

In this or other embodiments, when the wearer wears the ankle foot orthosis, the wearer is allowed to perform about a 45-degrees of maximum perform plantar flexion and about a 45-degrees of maximum dorsiflexion, but the disclosure is not limited thereto. Upon the detailed descriptions of the above embodiments, people skilled in the art would be able to exert a modification adjustment to obtain an ankle foot orthosis that permits a different amount of plantar flexion and dorsiflexion exercise.

Briefly, by changing the insertion position of the pin 330b, the movement limitation to the ankle is able to be altered so as to fit the requirements of the rehabilitation. Certainly, in other cases, the pin 330b may be inserted in to another sub insertion holes 3111b1 of the insertion hole 3111b so as to obtain other combinations of movement limitation in dorsiflexion and plantar flexion, and the principles are similar to the above examples, so the detailed descriptions would not be repeated. In other words, the ankle foot orthosis 1b is applicable to various stages of rehabilitation.

In addition, similar to the said ankle foot orthosis 1a of the first embodiment, the first pivoting mechanism 30b and the second pivoting mechanism 40b of the ankle foot orthosis 1b may be different in size so that the wear may visually figure out whether the ankle foot orthosis 1b is for the left foot or the right foot, reaching fool-proof.

Furthermore, in the above embodiments, the first and second connecting parts of the first pivoting mechanism may be switched places, further, the pin and the stopper may be switched places, and the engagement portions may be switches places as well. Such modifications would not depart from the spirit and scope of the disclosure. Similarly, the components on the third and fourth connecting parts of the second pivoting mechanism may also be switched places, and the disclosure is not limited thereto.

Lastly, it is explained that, the first and second pivoting mechanisms may be assembled by being placed into openings via the engagement portions and slide into grooves respectively in the above embodiments, but the disclosure is not limited by the shape or quantity of the above engagement portions and openings of the pivoting mechanisms; for example, in another embodiment, the shape of the engagement portions and the openings may be different from that in the above embodiments, and the quantity of the engagement portions and the openings may both be one. In addition, the above ankle foot orthosis all have two pivoting mechanisms at both sides, but the disclosure is not limited thereto; for example, in still another embodiment, the ankle foot orthosis may omit the second pivoting mechanism; in this case, the ankle foot orthosis is still able to be assembled/detached without tools, and the ankle foot orthosis is still able to limit the motion of the foot.

According to the ankle foot orthosis discussed above, since one of the first connecting part and the second connecting part of the first pivoting mechanism has engagement portions, and the other one has groove and openings connected to the groove and allowing the engagement portions to pass through, the first connecting part and the second connecting part can be detached by directly removing the engagement portions from the groove via the opening. Therefore, the ankle foot orthosis can be detached in an efficient manner. In other words, the first connecting part and the second connecting part of the first pivoting mechanism can be assembled by directly engaging the engagement portions into the groove via the openings, thereby assembling the ankle foot orthosis in a fast manner. Therefore, the ankle foot orthosis can be assembled or disassembled by the wearer in a fast and convenient manner without using additional tools, which is convenient to wear and detach individual components for cleaning by the wearer for enhanced hygiene and comfort.

In addition, since the pin located between the first stopper and the second stopper is able to limit the degree of pivotal movement of the first connecting part and the second connecting part to each other so as to limit the pivotal movement of the lower leg piece relative to the foot plate when wearing, dorsiflexion and plantar flexion at the ankle are restricted.

Further, the disassemble angles of the first pivoting mechanism and the second pivoting mechanism that are in the detachable state are different. This helps the user to disassemble the first pivoting mechanism and the second pivoting mechanism one at a time and helps to avoid the first pivoting mechanism and the second pivoting mechanism from falling apart at the same time during the disassembly process. In contrary, the assemble angles of the first pivoting mechanism and the second pivoting mechanism that are in the assemblable state are also different. This helps the user to assemble the first pivoting mechanism and the second pivoting mechanism one at a time, thereby preventing the inconvenience for assembling two opposite adjustment mechanism.

Furthermore, in one embodiment, similar to the first pivoting mechanism, the second pivoting mechanism may include a pin to further limit the pivotable movement of the third connecting part relative to the fourth connecting part, thereby strengthening the restriction in the pivotable movement.

Moreover, in one embodiment, the first pivoting mechanism may provide a plurality of combinations of movement limitation in dorsiflexion and plantar flexion, such that the ankle foot orthosis is applicable to various stages of rehabilitation, such as increasing the off-ground period of the foot during the swing phase, and improving the stability of the ankle during the stance phase, the locomotion, and muscle strength. Comparing to the conventional ankle foot orthosis which is unmovable, the ankle foot orthosis of the disclosure is more applicable and more cost-effective.

It will be apparent to those skilled in the art that various modifications and variations can be made to the present disclosure. It is intended that the specification and examples be considered as exemplary embodiments only, with a scope of the disclosure being indicated by the following claims and their equivalents.

What is claimed is:

1. An ankle foot orthosis, comprising:
a foot plate and a lower leg piece; and
a first pivoting mechanism, comprising a first connecting part, a second connecting part and a pin, the first connecting part connected to the foot plate, the second connecting part connected to the lower leg piece, the first connecting part having an engagement portion, the second connecting part having a groove and an opening connected to the groove, the engagement portion located in the groove so that the first connecting part is pivotably engaged with the second connecting part, allowing the lower leg piece to be pivotably connected to the foot plate, the pin detachably disposed on the first connecting part of the first pivoting mechanism, and the second connecting part having a first stopper and a second stopper spaced apart from each other so as to limit a movement range of the pin;
wherein, the first pivoting mechanism has a first detachable state; when the first pivoting mechanism is in the first detachable state, the engagement portion is located at the opening and is detachable from the groove via the opening.

2. The ankle foot orthosis according to claim 1, wherein when the first pivoting mechanism is not in the first detachable state, the pin is located between an inner area between the first stopper and the second stopper.

3. The ankle foot orthosis according to claim 2, wherein the lower leg piece is pivotable relatively to the foot plate about a central pivot axis, and a pivotable angle of the pin in the inner area between the first stopper and the second stopper is between 0 degree and 90 degrees.

4. The ankle foot orthosis according to claim 3, wherein the first pivoting mechanism further has an elastic portion extending from the second stopper to the first stopper.

5. The ankle foot orthosis according to claim 2, wherein the lower leg piece is pivotable relatively to the foot plate about a central pivot axis, the first connecting part has an insertion hole, the insertion hole has a plurality of sub insertion holes arranged along an arc-shaped direction and connected to one another, and the pin is detachably disposed in one of the plurality of the sub insertion holes.

6. The ankle foot orthosis according to claim 2, wherein the first connecting part has an insertion hole, the pin is detachably disposed in the insertion hole; when the first pivoting mechanism is in the first detachable state, at least part of the insertion hole is not located above the inner area between the first stopper and the second stopper; and when the first pivoting mechanism is not in the first detachable state, the insertion hole is located above the inner area between the first stopper and the second stopper.

7. The ankle foot orthosis according to claim 1, wherein the first connecting part has a pivot hole, and the second connecting part has a pivot pillar detachably located in the pivot hole.

8. The ankle foot orthosis according to claim 1, further comprises a second pivoting mechanism opposite to the first pivoting mechanism, the second pivoting mechanism comprises a third connecting part and a fourth connecting part that are respectively connected to the foot plate and the lower leg piece, the third connecting part has another engagement portion, the fourth connecting part has another groove and another opening that is connected to the another groove, the another engagement portion is located in the another groove so that the third connecting part is pivotably engaged with the fourth connecting part, wherein the second angle adjustment mechanism has a second detachable state; when the first pivoting mechanism is in the first detachable state, the foot plate has a first angle at the lower leg piece; when the second angle adjustment mechanism is in the second detachable state, the another engagement portion is located in the another opening, and the foot plate is has an second angle at the lower leg piece, and the second angle is different from the first angle.

9. The ankle foot orthosis according to claim 1, further comprising at least one instep strap, wherein two opposite ends of the at least one instep strap are respectively detachably connected to two opposite sides of the foot plate.

10. The ankle foot orthosis according to claim 1, further comprising at least one lower leg strap, wherein two opposite ends of the lower leg strap are respectively detachably connected to two opposite sides of the lower leg piece.

11. The ankle foot orthosis according to claim 1, further comprising an anti-slip pad located on a side of the foot plate facing away from the lower leg piece.

12. The ankle foot orthosis according to claim 1, wherein the foot plate has a heal cover configured to be taller than or equal to a calcaneus of a wearer.
